SEQUENCE LISTING (3 sequences)

SEQ ID NO: 1 - protein, 528 aa, Homo sapiens
MPTPDATTPQAKGFRRAVSELDAKQAEAIMVRGQGAPGPSLTGSPWPGTAAPAASYTPTPRSPRFIGRRQSLIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 2 - DNA, 1898 bp, Homo sapiens
cggacctccacactgagccatgcccacccccgacgccaccacgccacaggccaagggcttccgcagggccgtgtctgagctggacgccaagcaggcagaggccatcatgggcgccccggggcccagcctcacaggctctccgtggcctggaactgcagccccagctgcatcctacacccccaccccaaggtccccgcggttcattgggcgcaggcagagcctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaggtgcacggcgtccctgagggcccttcccaacctcccctggtcctgcactgtcccggagctcaggccctggtgaggggctgggtcccgggtgccccccatgccctccctgctgccaggctcccactgcccctgcacctgcttctcagcgcaacagctgtgtgtgcccgtggtgaggttgtgctgcctgtggtgaggtcctgtcctggctcccagggtcctgggggctgctgcactgccctccgcccttccctgacactgtctgctgccccaatcaccgtcacaataaaagaaactgtggtctcta

SEQ ID NO: 3 - protein, 450 aa, Homo sapiens
MREIVHIQAGQCGNQIGAKFWEVISDEHGIDPSGNYVGDSDLQLERISVYYNEASSHKYVPRAILVDLEPGTMDSVRSGAFGHLFRPDNFIFGQSGAGNNWAKGHYTEGAELVDSVLDVVRKECENCDCLQGFQLTHSLGGGTGSGMGTLLISKVREEYPDRIMNTFSVVPSPKVSDTVVEPYNATLSIHQLVENTDETYCIDNEALYDICFRTLKLATPTYGDLNHLVSATMSGVTTSLRFPGQLNADLRKLAVNMVPFPRLHFFMPGFAPLTARGSQQYRALTVPELTQQMFDAKNMMAACDPRHGRYLTVATVFRGRMSMKEVDEQMLAIQSKNSSYFVEWIPNNVKVAVCDIPPRGLKMSSTFIGNSTAIQELFKRISEQFTAMFRRKAFLHWYTGEGMDEMEFTEAESNMNDLVSEYQQYQDATAEEEGEMYEDDEEESEAQGPK

(19) United States
(12) Patent Application Publication
(10) Pub. No.: US 2020/0341017 A1
(43) Pub. Date: Oct. 29, 2020

(54) 3D CELL CULTURE

(71) Applicant: UNIVERSITÉ DU LUXEMBOURG, Esch-sur-Aizette (LU)

(72) Inventors: Silvia Bolognin, Luxembourg City (LU); Marie Fossepre, Arlon (BE); Paul Antony, Esch-sur-Alzette (LU); Jens Schwamborn, Trier (DE)

(21) Appl. No.: 16/757,161

(22) PCT Filed: Oct. 22, 2018
(86) PCT No.: PCT/EP2018/078882

§ 371 Date: Apr. 17, 2020

(30) Foreign Application Priority Data
Oct. 20, 2017 (EP) 17197574.1
Oct. 24, 2017 (LU) LU100488

(51) Int. Cl. G01N 33/94; C12N 5/0793; G01N 33/50; C12N 5/0797; C12Q 1/26

(52) U.S. Cl. CPC G01N 33/9413; C12N 5/0619; G01N 33/5058; C12N 5/0623; C12Q 1/26; C12N 2501/15; C12N 2533/90; C12N 2513/00; C12N 2506/45; C12N 2501/999; C12N 2501/41; G01N 2500/10

(57) ABSTRACT

The present invention relates to a method for identifying molecules promoting or inhibiting dopaminergic neuronal differentiation and/or death of dopaminergic neurons in a three-dimensional cell culture. Furthermore, the present invention relates to a method for producing dopaminergic neurons in a three-dimensional cell culture. In addition, the present invention relates to a method for segmenting an image of a cell culture.

### FIELD OF THE INVENTION

The present invention relates to a method for identifying molecules promoting or inhibiting dopaminergic neuronal differentiation and/or death of dopaminergic neurons in a three-dimensional cell culture. Furthermore, the present invention relates to a method for producing dopaminergic neurons in a three-dimensional cell culture.

### BACKGROUND

The identification of promising drug candidates in pre-clinical research is hampered by the lack of sufficiently representative in vitro models. This is particularly true in the case of Parkinson's disease (PD), a complex disease where the affected cells are a specific population, the dopaminergic neurons of the substantia nigra in the midbrain. PD is a human specific disease for which animal models are not predictive of the human response. In this scenario, human induced pluripotent stem cells (iPSCs) are the ideal starting cell population for the generation of relevant in vitro models, due to their ability to differentiate into any cell type of the body.

Although in standard cell culture conditions, phenotypes are detectable, they are weak, often not reproducible and do not allow to clearly identify the effect of potential drug candidates with high-throughput screening approaches.

The objective was to find a tool for improved drug screening. The technical problem can thus be seen in the provision of an improved in vitro culture of iPSC derived dopaminergic neurons (DNs).

The technical problem is solved by the embodiments reflected in the claims, described in the description, and illustrated in the Examples and Figures.

### SUMMARY OF THE INVENTION

The above being said, the present invention relates to a method for identifying molecules promoting or inhibiting dopaminergic neuronal differentiation and/or death of dopaminergic neurons in a three-dimensional cell culture, the method comprising
a) differentiating neuroepithelial stem cells (NESCs) in a differentiation medium, wherein the differentiation medium comprises
(i) a SHH-pathway activator;
(ii) at least two different neurotrophins, and
(iii) an antioxidant;
b) further differentiating the cells obtained in a) in a differentiation medium, wherein the differentiation medium comprises
(i) at least two different neurotrophins, and
(ii) an antioxidant;
c) adding a molecule of interest to the differentiation medium in a) and/or b), wherein an increase of the differentiation into dopaminergic neurons compared to a control indicates that the molecule of interest promotes dopaminergic neuronal differentiation and/or inhibits death of dopaminergic neurons and wherein a decrease of the differentiation into dopaminergic neurons compared to a control indicates that the molecule of interest inhibits dopaminergic neuronal differentiation and/or induces death of dopaminergic neurons.

The present invention also relates to a method for producing dopaminergic neurons in a three-dimensional cell culture, the method comprising
a) contacting neuroepithelial stem cells (NESCs) with a matrix and/or a scaffold and optionally further a maintenance medium;
b) plating the NESCs in a container, wherein the container does not comprise a mean for generating fluid flow, thereby forming a three dimensional gel comprising NESCs;
c) differentiating NESCs obtained in b) in a differentiation medium, wherein the differentiation medium comprises
(i) a SHH-pathway activator;
(ii) at least two different neurotrophins; and
(iii) an antioxidant;
d) further differentiating the cells obtained in c) in a differentiation medium, wherein the differentiation medium comprises
(i) at least two different neurotrophins; and
(ii) an antioxidant;

thereby differentiating said NESCs into dopaminergic neurons.

The present invention also relates to a dopaminergic neuron obtainable by a method of the present invention.

The present invention also relates to a dopaminergic neuron of the present invention use in the treatment of a subject, preferably a subject suffering from a neurodegenerative disease.

### THE FIGURES SHOW

FIG. 1. Pipeline of the study.

Representative scheme of the pipeline used. hNESC reprogrammed from fibroblasts of PD patients as well as from healthy individuals were seeded in 2D and 3D conditions. After immunofluorescence staining, neurons were imaged with the Opera high content screening system (Perkin Elmer). 20 planes were acquired for each culture and the images were then stitched in Matlab. Automated image analysis was performed to extract i) nuclei volume (based on Hoechst), ii) neuronal volume (Tuj1), iii) expression of the dopaminergic marker TH, iiii) semi-quantitative analysis of the expression levels of pS129-α-synuclein. Scale bar 100 μm. In a similar manner also the fragmentation and the levels of pS129αsynuclein were analyzed.

FIG. 2. Expression of the dopaminergic marker TH and phosphorylation level of pS129SNCA in IPSC-derived neurons from healthy individual and PD patients.

Expression levels of TH normalized on the levels of neurons, Tuj1, A) and fragmentation of TH positive neurons (B). Expression levels of pS129SNCA normalized on the levels of neurons, Tuj1, (C). Neurons were imaged after 6 weeks of neuronal differentiation. *p<0.05,**<0.01, n=3−4.

FIG. 3. Clustergram of a cell pair from an healthy individual (K7WT), where the G2019S mutation was inserted (K7Mut). Heat map and computed hierarchical clustering of dopaminergic neurons after 6 weeks of neuronal differentiation. The Inh2 was administered at 0.5 μM concentration, twice a week for 6 weeks. Automated image analysis was performed to extract i) nuclei volume (based on Hoechst), ii) neuronal volume (Tuj1), iii) expression of the dopaminergic marker TH, iiii) semi-quantitative analysis of the expression levels of pS129-SNCA. N=4.

FIG. 4. Clustergram of a cell pair derived from a PD patients (IM5Mut), where the G2019S mutation was corrected (IM5GC). Heat map and computed hierarchical clustering of dopaminergic neurons after 6 weeks of neuronal differentiation. The Inh2 was administered at 0.5 μM concentration, twice a week for 6 weeks. Automated image analysis was performed to extract i) nuclei volume (based on Hoechst), ii) neuronal volume (Tuj1), iii) expression of the dopaminergic marker TH, iiii) semi-quantitative analysis of the expression levels of pS129-SNCA. N=3.

FIG. 5. Pathological reduction of pS129-α-SNCA due to G2019S LRRK2 mutation and rescue after Inh2 treatment in a cell pair from an healthy individual (H), where the G2019S mutation was inserted (HMut). Expression levels of pS129-α-SNCA normalized on the levels of neurons (Tuj1) after 6 week of neuronal differentiation in a 3D (A) and 2D (B) system. *p<0.05,**<0.01, n=4.

FIG. 6. Process of image analysis.

FIG. 7. Tuj 1 staining of wildtype (WT) and mutant cells (G2019S LRRK2 mutation) in 2D and 3D cultures. The phenotype of the mutant cells can only be detected in the 3D culture, while the 2D culture appears to be the same as the control culture.

### DETAILED DESCRIPTION

The identification of promising drug candidates in pre-clinical research is hampered by the lack of sufficiently representative in vitro models, particularly for Parkinson's disease (PD). We have used iPSC lines for the generation of midbrain specific dopaminergic neurons and for the identification of cellular related phenotypes (Reinhardt et al. (2013) “Derivation and expansion using only small molecules of human neural progenitors for neurodegenerative disease modeling.” PLoS ONE, 8, e59252). Although in standard cell culture conditions, phenotypes are detectable, they are weak, often not reproducible and do not allow to clearly identify the effect of potential drug candidates with high-throughput screening approaches.

To study PD-specific phenotypes in the derived neuronal cultures, we have established a pipeline to generate midbrain specific dopaminergic neurons in 3D conditions to better simulate the physiological niche. The 3D environment provides a balance between accumulation of paracrine factors and renewal of nutrients in the extracellular matrix. The derivation of dopaminergic neurons in 3D has also the advantages of i) using low volumes of media and consequently less amounts of novel neuroactive substances used for the assay, and ii) the option for multiplexing (96-well plate format), which is essential for automated screening activities.

We have used iPSC lines from PD patients with the G2019S mutation in LRRK2 as well as from healthy control individuals. In the patient lines the mutation has been corrected to the wild-type sequence, while in the iPSC lines from healthy individuals the wild-type sequence has been replaced by the mutated one (isogenic cell line pairs). Here we use these cells as a general model for Parkinson's disease.

The present inventors have found the successful integration of advanced developmental cell biology and cell culture technology. Differentiated human iPSC derived neuroepithelial stem cells (Reinhardt et al. (2013) “Derivation and expansion using only small molecules of human neural progenitors for neurodegenerative disease modeling.” PLoS ONE, 8, e59252) can efficiently be differentiated into functional dopaminergic neuron within 3D cell culture without the need to use microfluidic cultures and/or bioreactors.

After 30 days to 6 weeks in culture, known phenotypic characteristics of dopaminergic neurons by immunofluorescence are present. 3D image analysis revealed that the 3D cultures the neurodegenerative phenotype resulting from the G2019S-LRRK2 mutation was much exacerbated compared to the phenotype observed in 2D cultures of the same cells. In particular, less TH was expressed by cells comprising the G2019S-LRRK2 mutation compared to (isogenic) control cells. Furthermore, a much pronounced DN neurodegeneration was observed in the 3D cultures when compared to the 2D cultures for cells that comprised the G2019S-LRRK2 mutation. In particular, a higher TH fragmentation, higher amounts of swollen dendrites were detected in 3D cultures compared to 2D cultures.

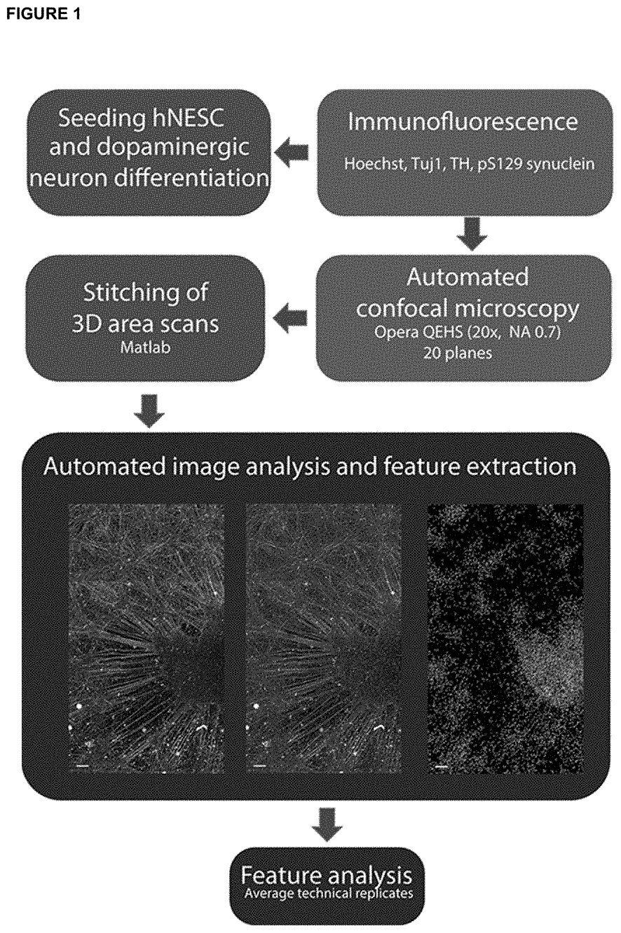

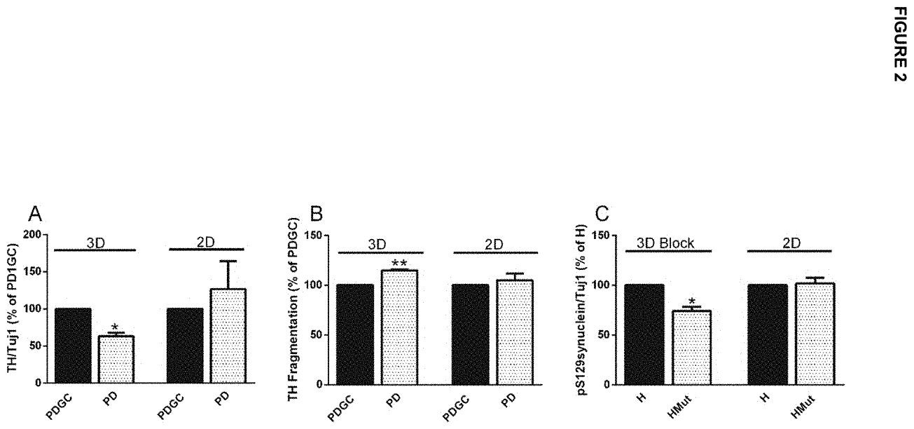

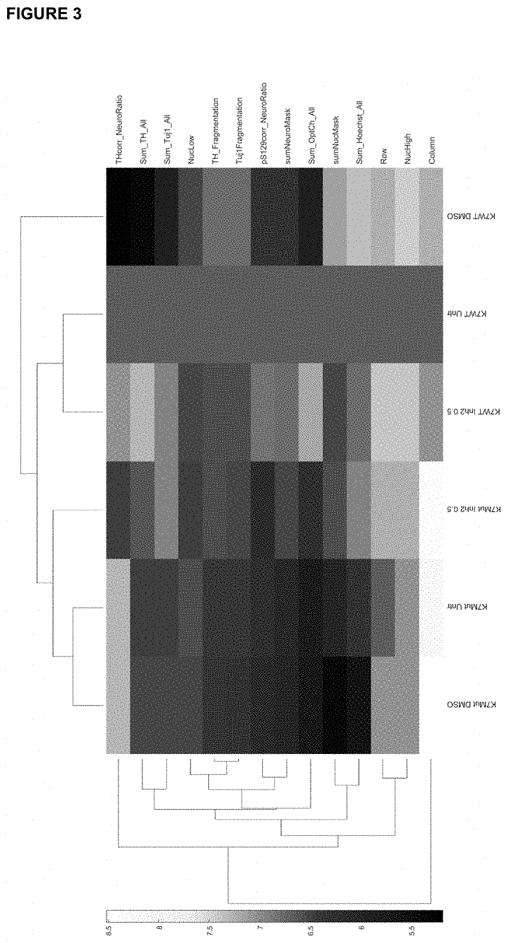

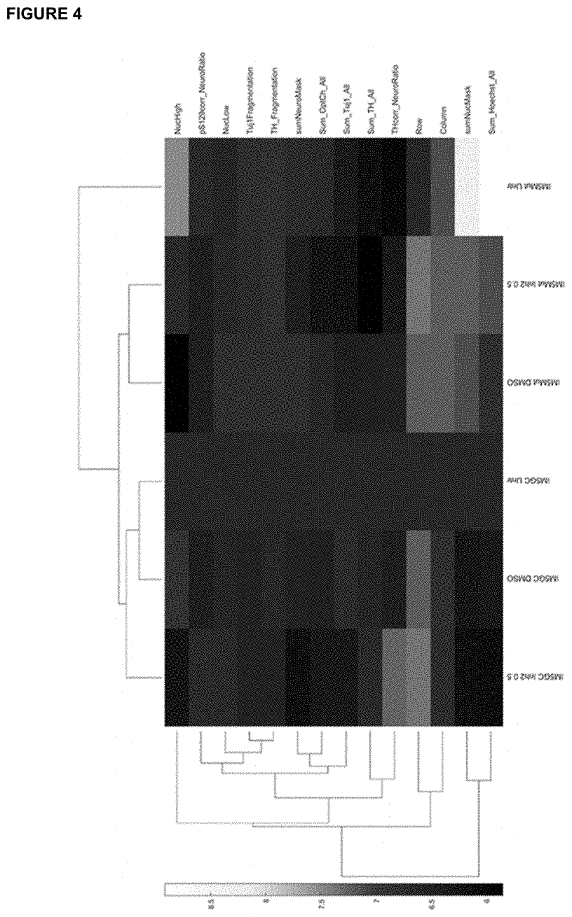

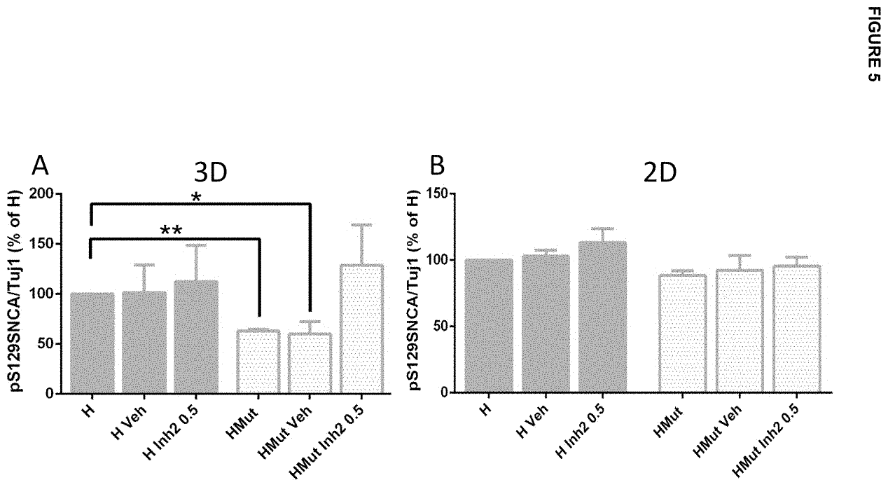

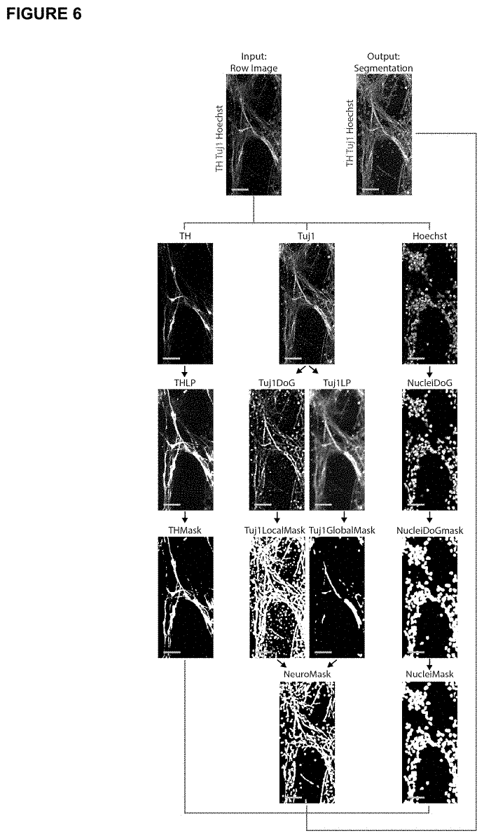

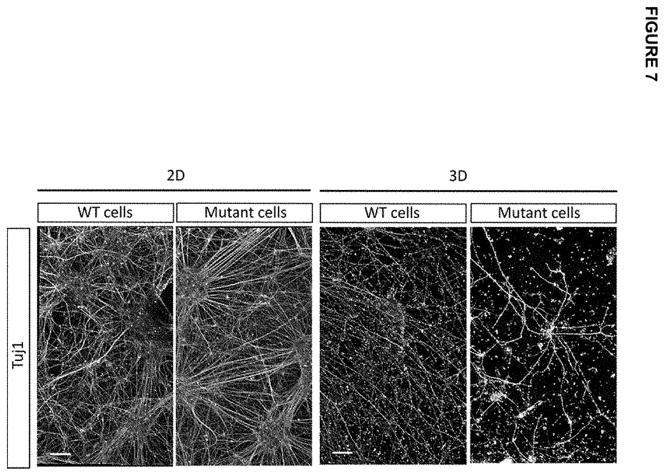

Thus, three dimensional cell culture without the need of microfluidic cell cultures or bioreactors can be successfully integrated with cellular reprogramming of neuroepithelial stem cells to produce an in vitro dopaminergic neuronal cell culture model. This model is robust, cost efficient, physiologically proximal and ready for parallelism by laboratory automation and personalization by supply of patient derived iPSC from dedicated bio-banks. Furthermore, cultures obtained in 3D cultures, without the utilization of microfluidic cell cultures and/or bioreactors are advantageous in terms of detecting a difference between the TH/Tuj cell population compared to a control (see FIG. 1).

Different methods to produce dopaminergic neurons from iPSCs are known to the skilled artesian. However, alternative methods that e.g. use less amounts of growth factors such as FGF and therefore are less cost-effective represent advantageous alternatives to known methods. Similarly, a method comprising fewer steps also represents an advantageous alternative to known methods. Accordingly, the methods of the present invention do not require the addition of FGF and therefore also lack a step of culturing cells in a medium comprising FGF compared to other methods as e.g. described in WO 2013/104752.

Furthermore, the inventors have surprisingly found that identifying molecules promoting or inhibiting dopaminergic neuronal differentiation and inhibiting death of dopaminergic neurons in a three-dimensional cell culture is advantageous compared to screening in two dimensional cultures. In two dimensional cultures, dopaminergic neurons e.g. derived from fibroblasts of patients afflicted with Parkinson's disease were similar to the dopaminergic neurons obtained from a healthy subject. Differences were primarily seen when these dopaminergic neurons were stressed by hydrogen peroxide (Nguyen et al., (2011) “LRRK2 mutant iPSC-derived DA neurons demonstrate increased susceptibility to oxidative stress.” Cell Stem Cell; 8(3):267-80).

Thus, the present invention provides for a method for producing dopaminergic neurons, or to a method for identifying molecules promoting or inhibiting dopaminergic neuronal differentiation and/or inhibiting death of dopaminergic neurons preferably the method is performed in a three-dimensional cell culture.

Furthermore, the methods are performable in stationary cultures not comprising fluid flow. This also eases the applicability of the methods of the present invention. Thus, the methods of the present invention are advantageous alternatives to other methods known in the art.

Similarly, dopaminergic neuronal death was hardly observed in two dimensional cultures of fibroblast-iPSC-derived neurons of patients afflicted with Parkinson's disease. In particular, an increase of 37% of TH and caspase 3 positive neurons was observed in cultures comprising fibroblast-iPSC-derived neurons of patients afflicted with Parkinson's disease compared to healthy subjects. Again, this phenotype only became more obvious upon stressing conditions (Reinhardt et al., (2013) “Derivation and expansion using only small molecules of human neural progenitors for neurodegenerative disease modeling.” PLoS One; 8(3):e59252).

From these data it was expected that also in three dimensional cultures differences between dopaminergic neurons derived from patients afflicted with Parkinson's disease and from control subjects would hardly differ or only show sharp differences upon stressing these cells.

In contrast to thereto the inventors have surprisingly found that in three dimensional cultures dopaminergic neurons derived from fibroblasts of different subjects (e.g. diseased and healthy subjects) can tremendously differ e.g. in their capacity to differentiate to dopaminergic neurons or to overt cell death. That the difference in differentiation and/or death is that obvious in 3D cultures could not be expected at all. Therefore, the present invention also provides for an advantageous screening method for identifying molecules promoting or inhibiting dopaminergic neuronal differentiation and death of dopaminergic neurons.

For image analysis software as described herein can be used. The software guaranties that the obtained results are much more reproducible than data obtained by a scientist counting by eye. The software principle can be applied to all data analyzed.

Thus, the present invention relates to a method for identifying molecules promoting or inhibiting dopaminergic neuronal differentiation and/or death of dopaminergic neurons in a three-dimensional cell culture, the method comprising
a) differentiating neuroepithelial stem cells (NESCs) in a differentiation medium, wherein the differentiation medium comprises
(i) a SHH-pathway activator;
(ii) at least two different neurotrophins, and
(iii) an antioxidant;
b) further differentiating the cells obtained in a) in a differentiation medium, wherein the differentiation medium comprises
(i) at least two different neurotrophins, and
(ii) an antioxidant;
c) adding a molecule of interest to the differentiation medium in a) and/or b), wherein an increase of the differentiation into dopaminergic neurons compared to a control indicates that the molecule of interest promotes dopaminergic neuronal differentiation and/or inhibits death of dopaminergic neurons and wherein a decrease of the differentiation into dopaminergic neurons compared to a control indicates that the molecule of interest inhibits dopaminergic neuronal differentiation and/or induces death of dopaminergic neurons.
Likewise in the method of the present invention an increase of the differentiation into dopaminergic neurons compared to a control indicates that the molecule of interest promotes dopaminergic neuronal differentiation and/or survival and wherein a decrease of the differentiation into dopaminergic neurons compared to a control indicates that the molecule of interest inhibits dopaminergic neuronal differentiation and/or survival.

The “control” as referred to herein can be any suitable control. The control can e.g. be the same culture without the addition of the molecule of interest. It may also be a culture of NESCs obtained from the very same subject without the addition or before the addition of the molecule of interest. The control may also be a culture obtained from NESCs obtained from a healthy subject or a subject not suffering from a neurodegenerative disease such as Parkinson's disease. It is also possible that the control is a subject comprising a LRRK2-G2019S mutation (Gilks et al. (2005), The Lancet 365 (9457): 415-416), which has been gene corrected by correcting the G2019S sequence into the wild-type sequence. This type of comparison has also been described in the Examples herein. Gene-correction is a method known to the skilled person and e.g. described in Reinhardt et al. (2013) “Genetic correction of a LRRK2 mutation in human iPSCs links parkinsonian neurodegeneration to ERK-dependent changes in gene expression.” Cell Stem Cell. 12(3):354-67 and Lee and Chung (2016) “Integrating Gene Correction in the Reprogramming and Transdifferentiation Processes: A One-Step Strategy to Overcome Stem Cell-Based Gene Therapy Limitations” Stem Cells Int.: 2725670).

Dopaminergic neuronal differentiation and/or death of dopaminergic neurons can be measured by different means. For example, the differentiation into dopaminergic neurons and their death can be measured by analyzing neurite outgrowth (see FIG. 7).

One advantage of the method of the present invention, when performed in a three dimensional culture is that, if neurite outgrowth is defect, there will be a strong phenotype visible in the three dimensional culture.

This means that in principle, for identifying molecules promoting or inhibiting dopaminergic neuronal differentiation and/or death of dopaminergic neurons in a three-dimensional cell culture, fibroblasts from a patient having Parkinson's disease can be differentiated into dopaminergic neurons. This will result in a neurite outgrowth in the three dimensional cell culture, which is decreased compared to a proper control culture (see FIG. 7). Molecules promoting or inhibiting dopaminergic neuronal differentiation and/or death of dopaminergic neurons can then be identified by their capacity to induce neurite outgrowth to an extend higher than the extend of neurite outgrowth in cultures obtained without the addition of a molecule of interest (control).

Similarly, also dopaminergic neurons obtained from fibroblasts of healthy subjects show extensive neurite outgrowth in a three dimensional culture. A molecule of interest can then be added to e.g. parallel cell cultures obtained from fibroblasts of the same subject. Molecules promoting or inhibiting dopaminergic neuronal differentiation and/or death of dopaminergic neurons can then be identified by their capacity to reduce neurite outgrowth compared to the neurite outgrowth in cultures obtained without the addition of a molecule of interest (control).

Therefore, the differentiation into dopaminergic neurons and/or dopaminergic neuronal death can be measured by comparing neurite outgrowth. The comparison can be performed with regard to a control e.g. between similar cultures e.g. from the very same subjects with and without the addition of the molecule of interest as described herein. However, also the comparison between a culture of a subject suffering from Parkinson's disease such as a LRRK2 mutation and a culture of a healthy subject is envisioned by the present invention.

Additionally or alternatively, differentiation into dopaminergic neurons and/or death of dopaminergic neurons can be measured by comparing the expression of Tyrosine 3-mono-oxygenase (TH). Tyrosine hydroxylase or tyrosine 3-monooxygenase is the enzyme responsible for catalyzing the conversion of the amino acid L-tyrosine to L-3,4-dihydroxyphenylalanine (L-DOPA). L-DOPA in turn is a precursor for dopamine, which neurotransmitter is present in dopaminergic neurons. [43] By detecting the expression of TH a cell can be verified to be a neuron. For example, the detection can comprise measuring the expression of a protein comprising a sequence of SEQ ID NO. 1 (TH protein sequence) or a protein comprising a sequence having 80%, 85%, 90%, 95%, 98%, 99% or 100% sequence identity to SEQ ID NO. 1.

Likewise TH expression may be detected on the nucleic acid molecule level. For example, the detection can comprise measuring the expression of a nucleic acid molecule comprising a sequence of SEQ ID NO. 2 (TH mRNA sequence) or a nucleic acid molecule comprising a sequence having 80%, 85%, 90%, 95%, 98%, 99% or 100% sequence identity to SEQ ID NO. 2.

Higher levels of TH expression when compared to a control indicate the presence of more dopaminergic neurons or more dopaminergic neurites/axons compared to a control. On the other hand lower levels of TH expression compared to a control indicate the presence of less dopaminergic neurons or less dopaminergic neurites. Similarly, higher levels of TH expression compared to a control indicate the presence of more dopaminergic neurons and greater survival/lesser death of dopaminergic neurons. On the other hand lower levels of TH levels of TH expression compared to a control indicate the presence of less dopaminergic neurons and lesser survival/greater death of dopaminergic neurons. Again comparison can be performed using the same culture with or without the molecule of interest or between different cultures e.g. of diseased and healthy subjects or e.g. of gene-corrected cells.

Additionally, or alternatively, differentiation into dopaminergic neurons and/or death of dopaminergic neurons can be measured by comparing the expression of (neuron specific) Class III 8-tubulin (TUBβIII also referred to as TuJ1 herein) present in newly generated immature postmitotic neurons and differentiated neurons and in some mitotically active neuronal precursors.

By detecting the expression of Tuj1 a cell can be verified to be a dopaminergic neuron. For example, the detection can comprise measuring the expression of a protein comprising a sequence of SEQ ID NO. 3 (Tuj1 protein sequence) or a protein comprising a sequence having 80%, 85%, 90%, 95%, 98%, 99% or 100% sequence identity to SEQ ID NO. 3.

Likewise e.g. Tuj1 expression may be detected on the nucleic acid molecule level. For example, the detection can comprise measuring the expression of a nucleic acid molecule comprising a sequence encoding for SEQ ID NO. 3 (Tuj1 protein sequence) or a nucleic acid molecule comprising a sequence coding for a protein having 80%, 85%, 90%, 95%, 98%, 99% or 100% sequence identity to SEQ ID NO. 3.

Higher levels of TUBβIII expression indicate the presence of more neurons or more neurites, which can be dopaminergic neurons (e.g. if they additionally express TH), compared to a control. On the other hand lower levels of TUBβIII expression indicate the presence of less neurons or less neurites. Similarly, higher levels of TUBβIII expression indicate the presence of more neurons and greater survival/lesser death of neurons, which can be dopaminergic neurons, compared to a control. On the other hand lower levels of TUBβIII levels of TUBβIII expression indicate the presence of less neurons and lesser survival/greater death of neurons, which can be dopaminergic neurons, compared to a control. Again comparison can be performed using the same culture with or without the molecule of interest or between different cultures e.g. of diseased and healthy subjects.

The methods as described herein can also comprise that the differentiation into dopaminergic neurons is measured by measuring the expression of TH or by measuring the expression of TH among Tuj1-positive neurons. In the former case e.g. the total number of TH-positive cells in the culture that comprises or comprised the compound of interest can be compared to the total number of TH-positive cells present in a control culture. In the latter case e.g. the total number of TH/Tuj-1-double positive cells in the culture that comprises or comprised the compound of interest can be compared to the total number of TH/Tuj-1-double positive cells present in a control culture.

In the methods described herein the death of dopaminergic neurons can additionally or alternatively be measured by measuring fragmentation of TH-positive neurons. For example, the fragmentation can be defined as the ratio between surface pixels and volume pixels.

Death of dopaminergic neurons can also be measured by other methods which are well-known to the skilled artesian. For example, cell cultures may be stained for caspase 3 or TUNEL. If the caspase 3 or TUNEL staining is increased in the test culture in comparison to the control culture (e.g. more cells out of all TH/Tuj1 double positive cells stain positive for caspase 3 or TUNEL compared to control) then the molecule of interest promotes dopaminergic cell death. On the contrary, when the caspase 3 or TUNEL staining is decreased in the test culture in comparison to the control culture (e.g. less cells out of all TH/Tuj1 double positive cells stain positive for caspase 3 or TUNEL compared to control) then the molecule of interest inhibits dopaminergic cell death.

The methods of the present invention are carried out in a three dimensional cell culture. A “three-dimensional cell culture” or “3D cell culture” as used herein means that cells are grown in an artificially-created environment in which cells are permitted to grow or interact with its surroundings in all three dimensions. This concept is known to the skilled artesian and for example described in Ravi et al. (2015) “3D cell culture systems: advantages and applications.” J Cell Physiol. 230(1):16-26 and Antoni et al. (2015) “Three-Dimensional Cell Culture: A Breakthrough in Vivo.” Int J Mol Sci. 16(3):5517-5527). To achieve the three dimensional property of the cell culture, cells are grown or differentiated in matrices or scaffolds. In principle, suitable matrices or scaffolds, which can be used in three dimensional cell cultures are known to the skilled artesian. Such matrices or scaffolds can therefore be any matrix or scaffold. For example, the matrix or scaffold can be an extracellular matrix comprising either natural molecules or synthetic polymers, a biological and synthetic hybrid, metals, ceramic and bioactive glass or carbon nanotubes.

Exemplary natural extracellular matrix molecules include collagen, basement membranes such as laminin or fibrin, alginates, chitosan, hyaluronic acid, silk fibroin, cellulose actetate, casein, chitin, fibrinogen, gelatine, elastin or poly-(hydroxyalkanoate). Synthetic extracellular matrix polymers include hyaluronic acid (HA) modified forms, poly-ethylen glycol (PEG) modified forms, self-assembling protein hydrogels, poly(lactic-co-glycolic acid) (PLGA), polycaprolactone (PCL), polyurethane or PGS. Biological and synthetic hybrids can for example include polycaprolactone-chitosan, PLLA-Hydroxyapatite, hydroxyapatite-bioglass-ceramic, poly-(hydroxylalkanoate)-bioglass, hydroxyapatite-collagen, PCL-gelatin or PCL-collagen. Exemplary metals include tantalam, magnesium and its alloys, titanium and its alloys or nitinol (nickel and titanium alloys). Examples of ceramics and bioactive glass matrices/scaffolds include titanium and tri calcium phosphate, hydroxyapatite and tricalcium phosphate, bioactive silicate glass (SiO₂—Na₂O—CaO—P₂O₅), hydroxyapatite and bioglass, calcium phosphate glass or phosphate glass. Carbon nanotubes can be constructed using graphite ranging from 0.4 to 2 nm. Carbon nanotubes can comprise CNT-polycaprolactone, CNT-ceramic matrix, 45S5 bioglass-CNT, CNT studded with gelatin hydrogel, CNT-TiO₂, CNT-laminin, CNT grafted with polyacrylic acid or CNT-TGF-β.

The matrix or scaffold can also be a hydrogel such as matrigel, fibrin gel or alginate gel. Matrigels can be a reconstituted basement membrane preparation extracted from Engelbreth-Holm-Swarm mouse sarcoma, a tumor rich in extracellular matrix proteins. Matrigel can be constituted of 60% laminin, 30% type IV collagen and 8% entactin. Optionally growth factors and other molecules can be added to the matrigel. The matrigel can also be mixed with a medium. E.g. the matrigel can be diluted with a medium as described herein or in the Examples. For example, the matrgel is diluted with a medium in a dilution ratio of 1:15. The matrigel can also be BD MatrigelTM (obtainable from BD Biosciences; catalogue number 354277).

It is further envisioned that the three-dimensional cell culture used in the present invention is not an organoid culture. Organoids are three-dimensional tissue structures, often generated from pluripotent stem cells (PSCs) but e.g. also from neuroepithelial stem cells, which self-organize and recapitulate complex aspects of their organ counterparts, ranging from physiological processes to regeneration and disease. Such organoids are known to the skilled person and inter alia described in Monzel et al. (2017) “Derivation of human midbrain-specific organoids from neuroepithelial stem cells” Stem Cell Reports vol. 8, 1144-1154 (midbrain organoids), Jo et al. (2016) “Midbrain-like organoids from human pluripotent stem cells contain functional dopaminergic and neuromelanin-producing neurons” Cell Stem Cell 19, 248-257 (midbrain organoids), or Quian et al. (2016) “Brain-region-specific organoids using mini-bioreactors for modeling ZIKV exposure” Cell 165, 771-785 (forebrain organoids) or Lancaster et al. (2013) “Cerebral organoids model human brain development and microcephaly” Nature 501, 373-379 (cerebral organoid).

It is further envisioned that the three dimensional culture is not a midbrain organoid. A midbrain organoid resembles the midbrain. The midbrain is the region of the brain, where the majority of the neurotransmitter dopamine (DA) is produced. A midbrain organoid thus comprises dopaminergic neurons. Such midbrain organoids are known to the skilled person and inter alia described in Monzel et al. (2017) “Derivation of human midbrain-specific organoids from neuroepithelial stem cells” Stem Cell Reports vol. 8, 1144-1154. In such midbrain organoids about 60% of the neurons are dopaminergic neurons. However, the amount of total neurons is rather low in such midbrain organoids, since also astrocytes (e.g. up to 4%) and oligodendrocytes (29.6% of all Tuj1-positive neurites are ensheathed by oligodendrocytes) are present in such a midbrain organoid (Monzel et al. (2017) “Derivation of human midbrain-specific organoids from neuroepithelial stem cells” Stem Cell Reports vol. 8, 1144-1154).

A further aspect that organoid cultures show is the spatial distribution of cells. However, the three-dimensional cell cultures used in the present invention do not show any such spatial organization of the cells resuming an organ.

In the three dimensional cell cultures used in the present invention about 20% of the neurons are dopaminergic neurons. Thus, it is also contemplated that in the three-dimensional cell culture less than about 50% of all neurons are dopaminergic neurons. It is further envisioned that in the three-dimensional cell culture less than about 45%, 40%, 35%, 30% 25% of all neurons are dopaminergic neurons.

In addition, the three dimensional cell cultures used in the present invention are mainly made up of neurons and only comprise low amounts of astrocytes or oligodendrocytes. Thus, it is further contemplated that in the three-dimensional cell cultures used in the present invention at least about 25% of all cells are neurons. It is further envisioned that in the three-dimensional cell culture at least about 25%, 30%, 35%, 40%, 45%, 50%, 55%, 60%, 65%, 70%, 75%, 80%, 85%, 90% of all cells present within the three-dimensional cell culture are neurons. It is further envisioned that about 80% or more of the total amount of cells in the three dimensional culture are neurons.

It is envisioned that the number of neurons with regard to all cells within a culture can inter alia be determined by a ratio of the total number of DAPI positive cells (for all cells present in the cell culture) divided by the total number of Tuj1-expressing cells (which are mature neurons).

It is further envisioned that in the number of dopaminergic neurons within all neurons can inter alia be determined by calculating a ratio of the total number of Tuj1-expressing cells (all neurons) divided by the total number of Tuj1 as well as tyrosine hydroxylase (TH) expressing cells (dopaminergic neurons).

It is also contemplated that the expression of Tuj1, dcx and/or TH can inter alia be determined by immunohistochemistry.

The methods as described herein comprise the differentiation of neuroepithelial stem cells (NESCs). Neuroepithelial cells are a class of stem cell and have similar characteristics as stem cells. For example, these cells are able to self-renew. Self-renewal is the ability to go through numerous cell cycles of cell division while maintaining the undifferentiated state. In addition, neuroepithelial stem cell cells have the capacity to differentiate further into multiple types of cells, such as neurons, astrocytes and other glial cells. Thus, these cells are also multipotent. Methods for testing if a cell has the capacity to self-renew and if a cell is multipotent are known to the skilled artesian. Self-renewal may be tested by passaging the cells over more than 10, 11, 12, 13, 14, 15, 16, 17, 18, 19, 20, 21, 22, 23, 24, 25, 26, 27, 28, 29, 30 or more passages. Passaging includes splitting of the cells before re-plaiting them as a single cell suspension. Multipotency can be tested by differentiating said cells into different lineages such as astrocytes, oligodendroctyes and neurons.

Furthermore, a neuroepithelial stem cell can express markers such as PAX6, Notch 1, Nestin, PCNA, HesS and Sox1. In particular, the neuroepithelial stem cells used in the methods of the present invention can be mammalian neural plate border stem cells (NPBSC) as described in WO2013104752. Furthermore, the neuroepithelial stem cells used in the methods of the present invention can also be NPBSCs as described in WO2013104752, which are also obtained by the method as described in WO2013104752. These NPBSC can be characterized by the expression of at least three markers selected from the group consisting of FORSE1, MSX1, PHOX2B, PAX3, PAX6, SOX1, SOX2, NESTIN, IRX3, HOXA2, HOXB2, HESS, DACH1, PLZF, LM03, EVI1 and ASCL1. Furthermore, these cells can be characterized by a lack of expression of at least one of the markers OCT4, NANOG, AFP, T, SOX17, EOMES, GSH2, OLIG2, CK8, CK18, NKX2.2, NKX6.1, HOXB8, HOXAS, FOXA2 and VCAM-1.

The neuroepithelial stem cell can be a mammalian NESC. It is also encompassed by the present invention that the NESC is a human NESC (hNESC). A neuroepithelial stem cell may be obtained by different means and methods known to the skilled artesian. NESCs can be derived from actual stem cells in several different stages of neural development. For example, a neuroepithelial stem cell may be derived or obtained from pluripotent cells.

A “pluripotent stem cell” when referred to herein relates to a cell type having the capacity for self-renewal, and the potential of differentiation into different cell types. Pluripotent stem cells can differentiate into nearly all cells, i.e. cells derived from any of the three primary germ layers: ectoderm, endoderm, and mesoderm. The term pluripotent stem cell also encompasses stem cells derived from the inner cell mass of an early stage embryo known as a blastocyst.

Notably, recent advances in embryonic stem cell research have led to the possibility of creating new embryonic stem cell lines without destroying embryos, for example by using a blastomere biopsy-based technique, which does not interfere with the embryo's developmental potential (Klimanskaya (2006) “Embryonic stem cells from blastomeres maintaining embryo viability.” Semin Reprod Med. 2013 Jan. 31 (1):49-55). Furthermore, a large number of established embryonic stem cell lines are available in the art. Thus it is possible to work with embryonic stem cells without the necessity to destroy an embryo. The pluripotent stem cells can be embryonic stem cells, which have not been obtained via the destruction of a human embryo. Thus, the pluripotent stem cells are embryonic stem cells obtained from an embryo, without the destruction of the embryo.

A neuroepithelial stem cell can also be derived or obtained from another pluripotent cell, namely an induced pluripotent stem cell (iPSC). “Induced pluripotent stem cells”, as used herein, refers to adult somatic cells that have been genetically reprogrammed to an embryonic stem cell-like state by being forced to express genes and factors important for maintaining the defining properties of embryonic stem cells. Thus, induced pluripotent stem cells can be derived from a non-pluripotent cell.

Induced pluripotent stem cells are an important advancement in stem cell research, as they allow obtaining pluripotent stem cells without the use of embryos. Mouse iPSCs were first reported in 2006 (Takahashi, K; Yamanaka, S (2006). “Induction of pluripotent stem cells from mouse embryonic and adult fibroblast cultures by defined factors”. Cell 126 (4): 663-76), and human iPSCs were first reported in 2007 (Takahashi et al. (2007) “Induction of pluripotent stem cells from adult human fibroblasts by defined factors.” Cell; 131(5):861-72). Mouse iPSCs demonstrate important characteristics of pluripotent stem cells, including expression of stem cell markers, forming tumors containing cells from all three germ layers, and being able to contribute to many different tissues when injected into mouse embryos at a very early stage in development. Human iPSCs also express stem cell markers and are capable of generating cells characteristic of all three germ layers. Such stem cell markers can include Oct3/4, Sox2, Nanog, alkaline phosphatase (ALP) as well as stem cell-specific antigen 3 and 4 (SSEA3/4). Also the chromatin methylation patterns of iPSC are also similar to that of embryonic stem cells (Tanabe, Takahashi, Yamanaka (2014) “Induction of pluripotency by defined factors.” Proc. Jpn. Acad., 2014, Ser. B 90).

In addition, iPSCs are able to self-renew in vitro and differentiate into all three germ layers. The pluripotency or the potential to differentiate into different cell types of iPSC can tested, e.g., by in vitro differentiation into neural or glia cells or the production of germline chimaeric animals through blastocyst injection.

Methods for the generation of human induced pluripotent stem cells are well known to the skilled person. Usually forced expression of Oct3/4, Sox2 and Klf4 (as well as OCT3/4, SOX2 and KLF4) is sufficient to generate an induced pluripotent stem cell out of an adult somatic cell, such as a fibroblast. However, also the combination of Oct3/4, Sox2, c-Myc and Klf4 (as well as OCT3/4, SOX2, C-MYC) and KLF4 is sufficient for the generation of an iPSC from an adult somatic cell. In addition also the combination of OCT3/4, SOX2, NANOG and LIN28 was efficient for reprogramming (Tanabe, Takahashi, Yamanaka (2014) “Induction of pluripotency by defined factors.” Proc. Jpn. Acad., 2014, Ser. B 90). For this, these genes are usually cloned into a retroviral vector and transgene-expressing viral particles or vectors, with which the somatic cell is co-transduced. However, also other techniques known to the skilled artesian can be used for that purpose. Human skin fibroblasts can also be co-transduced with all four vectors e.g. via protein transduction or naked DNA.

Further methods for obtaining iPSCs are also known to the skilled artesian and for example described in WO2009115295, WO2009144008 or EP2218778. Thus, the skilled artesian can obtain an iPSC by any method.

In principle, induced pluripotent stem cells may be obtained from any adult somatic cell (obtained from a subject). Exemplary somatic cells include peripheral blood Mononuclear Cells (PBMCs) from blood, keratinocyte, T-cell, CD34+ cell, myeloid cell, or a renal epithelial cell or fibroblasts, such as for example fibroblasts obtained from skin tissue biopsies.

Therefore, it is envisioned by the present invention that the NESC is produced or derived or obtained from an induced pluripotent stem cell (iPSC). The iPSC can for example be a human iPSC (hiPSC). Different ways how to differentiate iPCSs into neuroepithelial stem cells are known to the skilled artesian and for example described in WO2013/104752. In addition, it is envisioned by the present invention that the iPSCs can be produced from somatic cells such as fibroblasts. Furthermore, the iPSC can be a human iPSC (hiPSC).

It is further encompassed by the present invention that the iPSCs can be obtained from a subject suffering from a neurodegenerative disease.

It is further encompassed by the present invention that the iPSCs can be obtained from a fibroblast, keratinocyte, T-cell, CD34+ cell, myeloid cell, or a renal epithelial cell that have been obtained from a subject suffering from a neurodegenerative disease.

A “neurodegenerative disease” as used herein relates to any neurodegenerative disease. Non-limiting examples include Alzheimer's disease, Parkinson's disease, amyotrophic lateral sclerosis, Huntington's disease and frontotemporal dementia.

The term “subject” can also mean human or an animal. The subject can also be a subject suffering from a neurodegenerative disease e.g. Parkinson's disease. The subject may also carry a mutation associated with a neurodegenerative disease. Such mutations are known to the skilled artesian and for example described in Bertram and Tanzi (2005) “The genetic epidemiology of neurodegenerative disease” J Olin Invest. 115(6): 1449-1457.

Exemplary mutations include mutations in the Aβ precursor protein (APP) on chromosome 21, presenilin 1 (PSEN1) on chromosome 14, and presenilin 2 (PSEN2) on chromosome 1, α-synuclein (SNCA or PARK1); parkin (PRKN or PARK2); DJ-1 (DJ1 or PARK7); PTEN-induced putative kinase I (PINK1 or PARK6); and leucine-rich repeat kinase 2 or dardarin (LRRK2 or PARKS), tau (gene: MAPT), superoxide dismutase 1 (SOD1) or alsin (ALS2). In particular, the subject may be a subject comprising a LRRK2-G2019S mutation, which is associated with Parkinson's disease. This mutation is well known in the art and inter alia described in Bouhouche (2017) “LRRK2 G2019S Mutation: Prevalence and Clinical Features in Moroccans with Parkinson's Disease” Parkinson's Disease Volume 2017, p.1-7 and Goldwurm et al. (2005) “The G6055A (G2019S) mutation in LRRK2 is frequent in both early and late onset Parkinson's disease and originates from a common ancestor” Med Genet; 42; doi: 10.1136; jmg.2005.035568, pages 1-8.

The subject can also be a subject not suffering from a neurodegenerative disease such as Parkinson's disease. Also encompassed by the present invention is that the subject is a healthy subject. In such cases a mutation that can cause a neurodegenerative disease as described herein can be introduced into the cells obtained from this subject as also described in the Examples for the provision of an isogenic control. The subject can be a vertebrate, more preferably a mammal. Mammals include, but are not limited to, farm animals, sport animals, pets, primates, dogs, horses, mice and rats. A mammal can also be a human, dog, cat, cow, pig, mouse, rat etc. Thus, in one embodiment, the subject is a vertebrate. The subject can also be a human subject.

The methods as described herein comprise the differentiation of NESCs in a differentiation medium. The differentiation medium in step a) comprises a SHH-pathway activator. However, the differentiation medium in b) may not comprise a SHH-pathway activator.

The term “activator”, as used herein, is defined as a compound/molecule enhancing or achieving the activity of a target molecule or pathway. The activator may achieve this effect by enhancing or inducing the transcription of the gene encoding the protein to be activated and/or enhancing the translation of the mRNA encoding the protein to be activated. It can also be that the protein to be activated performs its biochemical function with enhanced efficiency in the presence of the activator or that the protein to be activated performs its cellular function with enhanced efficiency in the presence of the activator. Accordingly, the term “activator” encompasses both molecules/compounds that have a directly activating effect on the specific pathway but also molecules that are indirectly activating, e.g. by interacting for example with molecules that negatively regulate (e.g. suppress) said pathway. The activator can also be an agonist of the pathway to be activated. Methods for testing if a compound/molecule is capable to induce or enhance the activity of a target molecule or pathway is known to the skilled artesian. For example an activator of a SHH, WNT or other activator as described herein can be tested by performing Western Blot analysis of the amount of e.g. pathway effector proteins such as Gli proteins or LEF1 or TCF1 protein, respectively.

The compound/molecule that can be used as an activator can be any compound/molecule, which can activate the respective pathway or which inhibits a suppressor of the pathway to be activated. Exemplary activators can include suitable binding molecules directed e.g. against suppressors of a certain pathway.

For example, the binding molecule can be an antibody or a divalent antibody fragment comprising two binding sites with different specificities. Non limiting examples of such divalent antibody fragments include a (Fab)₂′-fragment, a divalent single-chain Fv fragment, a bsFc-1/2-dimer or a bsFc-CH3-1/2 dimer. Alternatively, the binding molecule can also be a bivalent proteinaceous artificial binding molecule such as a lipocalin mutein that is also known as “duocalin”. The binding molecule may also only have a single binding site, i.e., may be monovalent. Examples of monovalent binding molecules include, but are not limited to, a monovalent antibody fragment, a proteinaceous binding molecule with antibody-like binding properties. Examples of monovalent antibody fragments include, but are not limited to a Fab fragment, a Fv fragment, a single-chain Fv fragment (scFv) or an scFv-Fc fragment.

The binding molecule can also be a proteinaceous binding molecule with antibody-like binding properties. Exemplary but non-limiting proteinaceous binding molecules include an aptamer, a mutein based on a polypeptide of the lipocalin family, a glubody, a protein based on the ankyrin scaffold, a protein based on the crystalline scaffold, an adnectin, an avimer or a (recombinant) receptor protein.

The activator can also be a nucleic acid molecule, such as a RNA, siRNA, miRNA or a non-proteinaceous aptamer. Such an aptamer is an oligonucleic acid that binds to a specific target molecule. These aptamers can be classified as: DNA or RNA aptamers. They consist of (usually short) strands of oligonucleotides. Also the nucleic acid molecules may be used to suppress a repressor of a pathway to be activated.

It is also encompassed by the present invention that the activator is a small molecule or protein/polypeptide. Such a small molecule can have a low molecular weight of less than 900 daltons (da), less than 800 da, less than 700 da, less than 600 da or less than 500 da. The size of a small molecule can be determined by methods well-known in the art, e.g., mass spectrometry. So for example an activator of the SHH pathway can be purmorphamine, which is a small-molecule agonist developed for the protein Smoothened. Thus, the activator can also be an agonist of the pathway to be activated.

An activator may enhance or increase the pathway to be activated by 10%, 20%, 30%, 40%, 50%, 60%, 70%, 80%, 90%, 100% or more when compared to the activity of the pathway without the addition of the activator.

The “Hedgehog signaling pathway” or “SHH pathway” is well known in the art and has been described, for example, in Choudhry et al. (2014) “Sonic hedgehog signalling pathway: a complex network.” Ann Neurosci. 21(1):28-31. Hedgehog ligands, including, for example, Sonic hedgehog, Indian hedgehog, and/or Desert hedgehog, bind to the receptor, including, for example, Patched or the patched-smoothened receptor complex, which induces a downstream signaling cascade. Downstream target genes of SHH signaling include GLI1, GL12 and/or GL13. Accordingly, the term “activator of the Hedgehog signalling pathway” also refers to an activator of any one of the above recited molecules that form part of this signaling pathway.

Exemplary activators of the Sonic hedgehog (SHH) signaling include purmorphamine (PMA; 2-(1-Naphthoxy)-6-(4-morpholinoanilino)-9-cyclohexylpurine 9-Cyclohexyl-N-[4-(4-morpholinyl)phenyl]-2-(1-naphthalenyloxy), CAS No.: 483367-10-8), SHH, smoothened agonist (SAG; 3-chloro-N-[trans-4-(methylamino)cyclohexyl]-N-[[3-(4-pyridinyl)phenyl]methyl]-benzo[b]thiophene-2-carboxamide, CAS No.: 912545-86-9) and Hh-Ag 1.5 (3-chloro-4,7-difluoro-N-(4-(methylamino)cyclohexyl)-N-(3-(pyridin-4-yl) benzyl)benzo[b]thiophene-2-carboxamide; CAS No.: 612542-14-0). The SHH-pathway activator can therefore be purmorphamine. The SHH pathway activator can also be a recombinant or truncated form of SHH, which retains SHH pathway activating functions such as e.g. SHH C24II.

The SHH signaling pathway activator such as purmorphamine can be employed in a concentration of between about 0.25 μM and about 1 μM, more preferably between about 0.4 μM and about 0.5 μM, and most preferably the amount is about 1 μM.

The SHH signaling pathway activator such as SHH can also be employed between about 50 and about 1000 ng/ml. The SHH signaling pathway activator such as SHH C24II can also be employed in a concentration of about 10 and about 500 ng/ml. The SHH signaling pathway activator such as SAG can be employed in a concentration of about 1 and about 100 nM. The SHH signaling pathway activator such as Hh-Ag1.5 can also be employed in a concentration of about 1 and about 50 nM.

The differentiation media of step a) and also of step b) of the methods of the present invention comprise at least two different neurotrophins. The term “neurotrophins”, as used herein, relates to a family of proteins that regulate the survival, development, and function of neurons. Exemplary neurotrophins include Insulin-like growth factor 1 (IGF), Fibroblast growth factors (FGF), Transforming growth factor beta (TGF), Leukemia inhibitory factor (LIF), nerve growth factor (NGF), brain-derived neurotrophic factor (BDNF), neurotrophin-3 (NT-3), neurotrophin-4 (NT-4) as well as GDNF family of ligands and ciliary neurotrophic factor (CNTF). The GDNF family of ligands includes glial cell line-derived neurotrophic factor (GDNF), neurturin (NRTN), artemin (ARTN), and persephin (PSPN).

Accordingly, the term “at least two different neurotrophins” refers to two or more of the recited molecules. Preferably, the at least two different neurotrophins are BDNF and GDNF (Gene Symbols: BDNF and GDNF, respectively). BDNF can e.g. be the human BDNF protein of Uniprot/Swissprot accession no. P23560 (version 1 as of Oct. 31, 1991). GDNF can e.g. be the human GDNF protein of Uniprot/Swissprot accession no. P39905 (version 1 as of Jan. 31, 1995).

BDNF and GDNF can both independently from each other be employed in a concentration of between about 0.0001 and about 50 ng/pl each, more preferably between about 0.001 and about 25 ng/pl each, and most preferably the amount is about 0.001 ng/pl each. BDNF and GDNF may for example be obtained from Peprotech.

The differentiation medium of step a) and also of step b) of the methods of the present invention further comprises an antioxidant. An antioxidant is a molecule that inhibits the oxidation of other molecules. The terms “oxidation” and “antioxidant” are well known in the art and have been described, for example, in Nordberg J, Arnér E S. (2001) “Reactive oxygen species, antioxidants, and the mammalian thioredoxin system.” Free Radic Biol Med. 31(11):1287-312. In short, oxidation is a chemical reaction involving the loss of electrons or an increase in oxidation state. Oxidation reactions can produce free radicals. In turn, these radicals can start chain reactions. When the chain reaction occurs in a cell, it can cause damage or death to the cell. Antioxidants terminate these chain reactions by removing free radical intermediates, and inhibit other oxidation reactions. Accordingly, an antioxidant refers to an inhibitor of a molecule involved in cellular oxidative processes.

Exemplary antioxidants include ascorbic acid, superoxide dismutase 1, superoxide dismutase 2, superoxide dismutase 3, glutathione, lipoic acid, epigallocatechin gallate, curcumine, melatonin, hydroxytyrosol, ubiquinone, catalase, vitamin E or uric acid. Thus, the antioxidant can be ascorbic acid.

The antioxidant such as ascorbic acid can be utilized in an amount of about 50 μM to about 1 mM, or between about 100 μM and about 500 μM, or the amount is about 200 μM. The antioxidant such as superoxide dismutase 1, 2 or 3 can also be employed between about 10 and about 500 units/ml. The antioxidant such as glutathione can also be employed between about 1 and about 10 ng/pl. Lipoic acid can be employed between about 200 and about 1000 μM. The antioxidant such as epigallocatechin gallate can be employed between about 10 and about 100 μg/ml. The antioxidant such as curcumin can be employed between about 10 and about 100 μM. The antioxidant such as melatonin can be employed between about 10 and about 200 μM. The antioxidant such as hydroxytyrosol can be employed between about 10 and about 100 μM. The antioxidant such as ubiquinone can be employed between about 10 and about 50 μM. The antioxidant such as catalase can be employed between about 10 and about 500 units/ml. The antioxidant such as vitamin E can be employed between about 100 and about 1000 μM.

The methods of the present invention further comprise differentiating the cells obtained in a) in a differentiation medium, wherein the differentiation medium comprises
(i) at least two different neurotrophins as described herein; and
(ii) an antioxidant as described herein, thereby differentiating said NESCs into dopaminergic neurons.

The differentiation medium of step b) can also be termed second differentiation medium herein. It is further envisioned by the present invention that the differentiation medium in b) does not comprise a SHH-pathway activator.

The first and/or second differentiation medium (of step a) and/or b)) of the methods of the present invention can further comprise an activator of activin/transforming growth factor-β (TGF-β) signaling pathway. The activin/TGF-β signaling pathway is known in the art and for example described in Heldin, Miyazono and ten Dijke (1997) “TGF-bold beta signaling from cell membrane to nucleus through SMAD proteins.” Nature 390, 465-471. In short, receptor ligands, including, for example, TGFB1, TGFB2, TGFB3, ACTIVIN A, ACTIVIN B, ACTIVIN AB, and/or NODAL, bind to a heterotetrameric receptor complex consisting of two type I receptor kinases, including, for example, TGFBR2, ACVR2A, and/or ACVR2B, and two type II receptor kinases, including, for example, TGFBR1 , ACVR1 B, and/or ACVR1C. This binding triggers phosphorylation and activation of a heteromeric complex consisting of an R-smad, including, for example, SMAD2, and/or SMAD3, and a Co-smad, including, for example, SMAD4. Accordingly, the term “activator of the activin/TGF-β signaling pathway” refers to an activator of any one of the above recited molecules that form part of this signaling pathway.

Exemplary activators of the activin/TGF-β3 signaling pathway include TGFβ1, TGFβ2, TGFβ3, activin A, activin B, activin AB or nodal. Thus, the activator of activin/TGF-β signaling pathway can be TGFβ3. The activator of the activin/TGF-β signaling pathway such as TGFβ3 can be utilized in an amount of 0.0001 ng/μl to 0.1 ng/pl such as e.g. in an amount of 0.001 ng/pl.

The first and/or second differentiation medium (of step a) and/or b)) of the methods of the present invention can further comprise a cAMP analogue. Such cAMP analogs are compounds that have similar physical, chemical, biochemical, or pharmacological properties as the cyclic adenosine monophosphate (cAMP). cAMP is known to the skilled artesian and described in e.g. Fimia G M, Sassone-Corsi P. (2001) “Cyclic AMP signalling.” J Cell Sci; 114(Pt 11):1971-2.

Exemplary cAMP analogues include forskolin, 8-(4-chloro-phenylthio)-2′-O-methyladenosine-3′,5′-cyclic monophosphate (8CPT-2Me-cAMP), 8-Chloro-cAMP (8-Cl-cAMP), Bucladesine, Rp-adenosine .3., 5.,-cyclic monophosphorothioate sodium salt (Rp-cAMPS), Sp-8-hydroxyadenosine .3., 5.,-cyclic monophosphorothioate sodium salt (Sp-80H-cAMPS) and Rp8-hydroxyadenosine .3., 5.,-cyclic monophosphorothioate sodium salt (Rp-80H-cAMPS) or dbcAMP. Thus, the cAMP analogue can be dbcAMP.

The first and/or second differentiation medium (of step a) and/or b)) of the methods of the present invention can further be a N2B27 medium (into which the different compounds are diluted). This means that the medium comprises a N2 supplement and a B27 supplement. Both supplements are well known to the person skilled in the art and freely available. The B27 supplement can be a B27 supplement without vitamin A. This B27 can be used at a concentration of 1:10-1:1000, such as 1:100 (supplement:medium). The B27 supplement can for example be obtained from Invitrogen. Likewise, also the N2 supplement can for example be obtained from Invitrogen. The N2 supplement may be used at a concentration of 1:20 to 1:2000, such as 1:200 (supplement:medium).

The differentiation medium can also be a Neurobasal medium and/or a DMEM-F12 medium. Both media can for example be obtained from Gibco. The N2B27 medium can for example comprise equal amounts of Neurobasal medium and DMEM/F12 medium.

The first and/or second differentiation medium (of step a) and/or b)) of the methods of the present invention can further comprise an antibiotic. Such an antibiotic can for example be a mix of penicillin and streptomycin. These antibiotics can be present at a concentration of 0.3%, 0.5%, 0.7%, 1%, 1.3%, 1.5%, 1.7%, 2%, 3%, 4%, 5% or more. Thus, the antibiotic such as a mix of penicillin and streptomycin can be present in a total concentration of 1% (including both penicillin and streptomycin).

The first and/or second differentiation medium (of step a) and/or b)) of the methods of the present invention can further comprise glutamine. For example, the differentiation media can further comprise L-glutamine. L-glutamine can be added at a concentration of 0.5 mM, 1mM, 1.5 mM, 2 mM, 2.5 mM 3 mM 3.5 mM, 4 mM or more. Thus, L-glutamine can be added at a concentration of 2 mM.

Thus, the differentiation medium in step a) and b) of the methods of the present invention can comprise N2B27 medium comprising about 50% DMEM-F12 (e.g. from Gibco)/about 50% Neurobasal (e.g. from Gibco), about 1:200 N2 supplement (e.g. from Invitrogen), about 1:100 B27 supplement lacking vitamin A (e.g. from Invitrogen), 1% Penicillin/Streptomycin and 2 mM L-glutamine (e.g. from Invitrogen).

It is also encompassed by the methods of the present invention that the differentiation is performed by
a) differentiating neuroepithelial stem cells (NESCs) in a differentiation medium, wherein the differentiation medium comprises

(i) a SHH-pathway activator;
(ii) at least two different neurotrophins;
(iii) an antioxidant;
(iv) an activin/TGF-β signaling pathway activator; and
(v) a cAMP analogue, and
b) further differentiating the cells obtained in a) in a differentiation medium, wherein the differentiation medium comprises
(i) at least two different neurotrophins;
(ii) an antioxidant;
(iii) an activin/TGF-6 signaling pathway activator; and
(iv) a cAMP analogue, and
thereby differentiating said NESCs into dopaminergic neurons.

Notably, it is also envisioned by the present invention that the differentiation medium in step a) and b) of the methods of the present invention does not comprise Fibroblast growth factor 8 (FGF8).

The method also encompasses that the cells are kept for 1, 2, 3, 4, 5, 6, 7, 8, 9, 10, 11, 12 days, preferably for 6 days, in the differentiation medium of step a).

The methods of the present invention also encompass that the cells can be kept for 1, 2, 3, 4, 5, 6, 7, 8, 9, 10, 11, 12 days in the differentiation medium of step a) of the methods of the present invention. Thus, the cells can be kept for 6 days (approx. 144 hours) in the differentiation medium of step a). It is further envisioned that the differentiation medium can be changed every 1, 2, 3, 4 or more days. Thus, the differentiation medium can be changed every 2 or 4 days. It is also encompassed by the methods of the present invention that the cells can be kept for 14, 15, 16, 17, 18, 19, 20, 21, 22, 23, 24, 25, 26, 27 or more days in the differentiation medium of step b) of the methods of the present invention. Thus, the cells are kept for 42 days (approx. 1008 hours) in the differentiation medium of step b)

Also in this step, the differentiation medium can be changed every 1, 2, 3, 4 or more days. Thus, the differentiation medium can be changed every 2 days. The differentiation medium can be changed every 4 days.

It is also encompassed by the methods of the present invention that the differentiation is performed by
a) differentiating neuroepithelial stem cells (NESCs) for about 4-7 days (approx. 96-168 hours), preferably 6 days in a differentiation medium, wherein the differentiation medium comprises
(i) a SHH-pathway activator;
(ii) at least two different neurotrophins;
(iii) an antioxidant;
(iv) an activin/TGF-(3 signaling pathway activator; and
(v) a cAMP analogue, and
b) further differentiating the cells obtained in a) for about 18-50 days, preferably 42 days in a differentiation medium, wherein the differentiation medium comprises
(i) at least two different neurotrophins;
(ii) an antioxidant;
(iii) an activin/TGF-6 signaling pathway activator; and
(iv) a cAMP analogue, and thereby differentiating said NESCs into dopaminergic neurons.

It is further encompassed by the present invention that before NESC cells are differentiated in differentiation medium they are maintained in a maintenance medium. Such a maintenance medium can comprise
(i) a SHH-pathway activator;
(ii) a canonical WNT-signaling activator; and
(iii) an antioxidant.

The cells can be kept for 1, 2, 3, 4, 5, 6, 7, 8, 9, 10 or more days in the maintenance medium. For example, the cells can be kept for one day in the maintenance medium. The cells can also be kept for two days in the maintenance medium.

The canonical Wnt signaling pathway is known to the skilled artesian and for example described in Logan and Nusse (Annu. Rev. Cell Dev. Biol. (2004) 20:781-810). In short, a Wnt ligand binds to Frizzled receptors, which triggers displacement of the multifunctional kinase GSK-3β from a regulatory APC/Axin/GSK-3β-complex. In the absence of Wnt-signal (Off-state), β-catenin, is targeted by coordinated phosphorylation by CK1 and the APC/Axin/GSK-3β-complex leading to its ubiquitination and proteasomal degradation through the β-TrCP/SKP pathway. In the presence of Wnt ligand (On-state), the co-receptor LRPS/6 is brought in complex with Wnt-bound Frizzled. This leads to activation of Dishevelled (Dvl), which displaces GSK-3β from APC/Axin. The transcriptional effects of Wnt ligand is mediated via Rac1-dependent nuclear translocation of β-catenin and the subsequent recruitment of LEF/TCF DNA-binding factors as co-activators for transcription. Exemplary Wnt ligands include for example Wnt1, Wnt3, Wnt3a, Wnt4, Wnt5a, Wnt7a, Wnt7b and/or Wnt11.

Accordingly, the term “canonical WNT-signaling activator” as described herein refers to an activator of any one of the above recited molecules that form part of this signaling pathway.

Exemplary canonical WNT-signaling activators include Norrin, R-spondin 2 or WNT protein. However, the canonical WNT-signaling activator can also block Axin or APC. This can be achieved for example via siRNA or miRNA technology. It is also encompassed by the present invention that the canonical WNT-signaling activator is a GSK-3 inhibitor. Exemplary GSK-3 inhibitors include CHIR 99021 (6-[[2-[[4-(2,4-Dichlorophenyl)-5-(5-methyl-1 H-imidazol-2-yl)-2-pyrimidinyl]amino]ethyl]amino]-3-pyridinecarbonitrile; CAS No.: 252917-06-9), SB-216763 (3-(2,4-Dichlorophenyl)-4-(1-methyl-1H-indo-3-yl)-1H-pyrrole-2,5-dione; CAS No.: 280744-09-4), 6-bromoindirubin-3′-oxime (CAS No.: CAS 667463-62-9), Tideglusib (4-Benzyl-2-(naphthalen-1-yl)-1,2,4-thiadiazolidine-3,5-dione), GSK-3 inhibitor 1 (CAS No.: 603272-51-1), AZD1080 (CAS No.: 612487-72-6), TDZD-8 (4-Benzyl-2-methyl-1,2,4-thiadiazolidine-3,5-dione; CAS No.: 327036-89-5), TWS119 (3-[[6-(3-aminophenyl)-7H-pyrrolo[2,3-d]pyrimidin-4-yl]oxy]-phenol; CAS No.: 601514-19-6), CHIR-99021 (CAS No.: 252917-06-9), CHIR-98014 (N6-[2-[[4-(2,4-dichlorophenyl)-5-(1H-imidazol-1-yl)-2-pyrimidinyl]amino]ethyl]-3-nitro-2,6-Pyridinediamine; CAS No.: 252935-94-7), SB 415286 (3-[(3-Chloro-4-hydroxyphenyl)-amino]-4-(2-nitrophenyl)-1 H-pyrrol-2,5-dione; CAS No.: 264218-23-7), LY2090314 (3-(9-fluoro-2-(piperidine-1-carbonyl)-1,2,3,4-tetrahydro-[1,4]diazepino[6,7,1-hi]indol -7-yl)-4-(imidazo[1,2-a]pyridin-3-yl)-1H-pyrrole-2,5-dione, CAS No.: 603288-22-8), AR-A014418 (N-(4-Methoxybenzyl)-N′-(5-nitro-1,3-thiazol-2-yl)urea; CAS No.: 487021-52-3 and/or IM-12 (3-(4-Fluorophenylethylamino)-1-methyl-4-(2-methyl-1H-indol-3-yl)-1H-pyrrole-2,5-dione; CAS No.: 1129669-05-1). Thus, the GSK-3 inhibitor can also be CHIR 99021.

The maintenance medium can comprise a N2B27 medium as described herein.

The maintenance medium can be used to prepare NESCs to prepare NESCs at a density of 16000 cells per 96-well plate well. It is further envisioned that the NESCs present in maintenance medium are mixed with a matrix as described herein such as Matrigel. It is further envisioned that the NESCs are contacted with a dilution ratio of 1:15 of matrix such as Matrigel and maintenance medium. The matrix can for example be Corning Matrigel hESC-qualified matrix catalogue number 354277 from Discovery Labware.

The present invention also relates to a method for producing dopaminergic neurons in a three-dimensional cell culture, the method comprising
a) contacting neuroepithelial stem cells (NESCs) with a matrix or a scaffold and optionally further a maintenance medium;
b) plating the NESCs in a container, wherein the container does not comprise a mean for generating fluid flow, thereby forming a three dimensional gel comprising NESCs;
c) differentiating NESCs obtained in b) in a differentiation medium, wherein the differentiation medium comprises
(i) a SHH-pathway activator;
(ii) at least two different neurotrophins; and
(iii) an antioxidant;
d) further differentiating the cells obtained in c) in a differentiation medium, wherein the differentiation medium comprises
(i) at least two different neurotrophins; and
(ii) an antioxidant;
thereby differentiating said NESCs into dopaminergic neurons.

It is envisioned that the container does not comprise an electronic device. Such an electronic device can be a pump. Additionally or alternatively, the container does not comprise a mechanic element. The mechanic element can be an enclined plane. It is further envisioned that the differentiation is not performed in a microfluidic cell culture. It is also envisioned that the differentiation is not performed in a bioreactor. What has been described for the method for screening herein applies mutatis mutandis to the method for producing a dopaminergic neuron and the the uses and dopaminergic neurons as described herein.

The pictures taken as described in the Examples herein can be analyzed via Matlab Software.

For the analysis of pictures e.g. via Matlab Software a method for segmenting an image of a cell culture as described herein can be applied. The method may be used to segment content of an image acquired by a microscope into functional groups, i.e. into sets of pixels defining certain structures. Prior to the acquisition of the image, the cell culture is dyed with different dyes to selectively stain structures thereof. For example, the first dye may be TH which is known to selectively stain axons in a specific neuronal subtype, optionally dopaminergic neural cells, the second dye may be Hoechst which is known to selectively stain nuclei of cells and the third dye may be Tuj1 which is known to selectively stain neural cells. The fluorescence of each dye is spectrally separated from the emission of the other two dyes, such that the image may be subdivided into separate layers or channels, each one comprising spectral emissions of one of the dyes. It is noted that dyes other than the ones mentioned may be used for the purposeful staining of structures in the cell culture. It is also envisioned that each dye fluoresces at a different wavelength as e.g. the case for Cy3 and Cy5.

It is further encompassed that the first dye can be selected from TH, DAT, FOXA2, GIRK2, Nurr1 or LMX1B (dopaminergic neurons). It is further encompassed that the second dye can be selected from Hoechst 33342, DAPI (D1306, D3571, D21490) or BOBO-1.

It is further encompassed that the third dye can be selected from doublecortin, NeuroD1, TBR1, beta III tubulin (Tuj1) or stathmin (immature neurons). Alternatively the third dye can be selected from NeuN, MAP2, 160kDa neurofilament medium, 200 kDa neurofilament heavy, synaptophysin or PSD95 (mature neurons).

All markers as described herein are known to the skilled artesian and can e.g. be obtained from abcam or other well-known biotech companies.

The method for segmenting an image of a cell culture may be used to analyze images of human neural epithelial stem cell (hNESCs) derived neuronal cultures and may be advantageously used in the field of immunofluorescence. The images may be acquired with a high content screening microscope such as Opera (PerkinElmer) at 20x lens magnification. Skew analysis may be performed before every plate acquisition in order to facilitate the subsequent image processing of acquired images. The acquired images may be segmented using the method as described herein which may be implemented in a numerical computing environment such as Matlab®. The method segmenting an image of a cell culture according to various embodiments may be seen to automate three major steps, namely: segmentation of nuclei, segmentation of neurons and feature extraction.

For the analysis of the first dye channel of the acquired image in order to identify the first group of pixels, raw data obtained from the image acquisition and corresponding to the fluorescence of the first dye may be processed by low-pass filtering, e.g. by convoluting the image with a Gaussian filter of size 10 and standard deviation 1 (THLP) and by subsequently thresholding the resulting image at a predefined pixel value, e.g. 100 (THMask).

For the analysis of the second dye channel of the acquired image which aims at segmentation of nuclei a difference of two processed second dye channel images may be calculated, such as the difference of two Gaussians (NucleiDoG). For example, a foreground image may be computed by convolving the raw second dye channel with a Gaussian filter of size 10 and standard deviation 2. Similarly, a background content of the image may be determined by applying a Gaussian filter of size 60 and standard deviation 20 to the second dye channel of the acquired image. In the difference image obtained by subtracting the background content from the foreground content, a nuclei mask may be defined by those pixels with values larger than 10 (NucleiDoGmask). In order to remove small autofluorescent structures in the image which can be excluded based on their size and would otherwise add noise if not removed, only pixels belonging to connected components/structures with a certain size may be retained in the second group of pixels. For example, a threshold size of 200 pixels may be used (NucleiMask). Since reduced noise increases the signal to noise ratio, these steps also aim to add sensitivity to the assay.

In order to analyze the third dye channel in order to segment neurons and thus to ultimately determine the third group of pixels, a combination of global and local thresholding is used according to various embodiments of the method. For global thresholding, the third dye channel of the image may be low-pass filtered (Tuj1LP), for example by convolving with a Gaussian filter of size 10 and standard deviation 3. Then, thresholding may be applied to the processed image, for example at a threshold of 150 (Tuj1GlobalMask). For local thresholding, a difference of two processed third dye channel images may be calculated such as the difference of two Gaussians (Tuj1DoG), wherein one processed image represents the foreground and the other processed image represents the background. For example, the background may be defined via convolution with a Gaussian filter of size 20 and standard deviation 6 and may be subtracted from the foreground defined via convolution of the image with a Gaussian filter of size 10 and standard deviation 3. Then, thresholding may be applied, wherein only pixels with a value above a predefined threshold are kept (Tuj1LocalMask). The threshold may be low, such as 3. The application of global and local thresholding to the third dye channel of the image may then be combined by forming the third group of pixels in which pixels are retained which were detected by either local or global thresholding.

The thus formed neuronal mask may be further refined by removing pixels therefrom which overlap with pixels of the second group of pixels. This step may be advantageous in that it removes pixels from the third group of pixels which are erroneously visible in the image although belonging to layers of cells in the 3D cell structure arranged above or below the examined cell layer. The examined cell layer may be defined by the confocal plane of the microscope as set during acquisition of the image of the cell culture.

In addition, as in the case of the second group of pixels, only pixels belonging to connected components/structures with a certain size may be retained in the third group of pixels. For example, a threshold size of 200 pixels may be used (NeuroMask). Since reduced noise increases the signal to noise ratio, these steps also aim to add sensitivity to the assay.

It the above description specific values for thresholds, sized and standard deviations of Gaussian filters have been given. It is noted that those values have been provided by way of an specific working example and should not be construed as limiting as they relate to a specific setup, as described above, that has been used by the inventors. The values may be chosen differently for other setups. It is further noted that the size of a Gaussian filter refers to its mask size, i.e. the size of the Gaussian kernel function in pixels as used in the image processing environment.

The present invention is further characterized by the following items:

1. Method for identifying molecules promoting or inhibiting dopaminergic neuronal differentiation and/or death of dopaminergic neurons in a three-dimensional cell culture, the method comprising
a) differentiating neuroepithelial stem cells (NESCs) in a differentiation medium, wherein the differentiation medium comprises
(i) a SHH-pathway activator;
(ii) at least two different neurotrophins, and
(iii) an antioxidant;
b) further differentiating the cells obtained in a) in a differentiation medium, wherein the differentiation medium comprises
(i) at least two different neurotrophins, and
(ii) an antioxidant; and
c) adding a molecule of interest to the differentiation medium in a) and/or b), wherein an increase of the differentiation into dopaminergic neurons compared to a control indicates that the molecule of interest promotes dopaminergic neuronal differentiation and/or inhibits death of dopaminergic neurons and wherein a decrease of the differentiation into dopaminergic neurons compared to a control indicates that the molecule of interest inhibits dopaminergic neuronal differentiation and/or induces death of dopaminergic neurons.

2. Method of item 1, wherein the differentiation into dopaminergic neurons is measured by measuring the expression of TH or by measuring the expression of TH among Tuj1-positive neurons.

3. Method of item 1 or 2, wherein the death of dopaminergic neurons is measured by measuring fragmentation of TH-positive neurons.

4. Method of any one of the preceding items, wherein the differentiation into dopaminergic neurons is measured by measuring the expression of TUBβIII.

5. Method of item 1, wherein the differentiation into dopaminergic neurons is measured by measuring neurite outgrowth.

6. Method of any one of the preceding items, wherein the NESC is a human NESC (hNESC).

7. Method of any one of the preceding items, wherein the NESC is obtained from an induced pluripotent stem cell (iPSC).

8. Method of any one of the preceding items, wherein the iPSC is a human iPSC (hiPSC).

9. Method of any one of the preceding items, wherein the iPSCs have been obtained from a peripheral blood Mononuclear Cells (PBMCs) from blood, keratinocyte, T-cell, CD34+ cell, myeloid cell, or a renal epithelial cell or fibroblasts, preferably the iPSCs have been obtained from a fibroblast.

10. Method of any one of the preceding items, wherein the iPSCs have been obtained from a peripheral blood Mononuclear Cells (PBMCs) from blood, keratinocyte, T-cell, CD34+ cell, myeloid cell, or a renal epithelial cell or fibroblasts, preferably the iPSCs have been obtained from a fibroblast of a subject suffering from a neurodegenerative disease.

11. Method of any one of the preceding items, wherein the neurodegenerative disease is selected from the group consisting of Alzheimer's disease, Parkinson's disease, amyotrophic lateral sclerosis, Huntington's disease and frontotemporal dementia.

12. Method of any one of the preceding items, wherein the NESCs have been obtained from a human subject suffering from Parkinson's disease e.g. from familial Parkinson's disease.

13. Method of any one of the preceding items, wherein the differentiation medium in b) does not comprise a SHH-pathway activator.

14. Method of any one of the preceding items, wherein the SHH-pathway activator is selected from the group consisting of purmorphamine, SHH, smoothened agonist (SAG) and Hh-Ag 1.5.

15. Method of any one of the preceding items, wherein the SHH-pathway activator is purmorphamine.

16. Method of any one of the preceding items, wherein the at least two neurotrophins are selected form the group consisting of IGF, FGF, TGF, LIF, NGF, BDNF, NT-3, NT-4, CNTF or GDNF.

17. Method of any one of the preceding items, wherein the at least two neurotrophins are GDNF and BDNF.

18. Method of any one of the preceding items—wherein the antioxidant is selected from the group consisting of ascorbic acid, superoxide dismutase 1, superoxide dismutase 2, superoxide dismutase 3, glutathione, lipoic acid, epigallocatechin gallate, curcumine, melatonin, hydroxytyrosol, ubiquinone, catalase, vitamin E or uric acid.

19. Method of any one of the preceding items, wherein the antioxidant is ascorbic acid.

20. Method of any one of the preceding items, wherein the differentiation medium further comprises an activator of activin/transforming growth factor-β (TGF-β) signaling pathway.

21. Method of any one of the preceding items, wherein the activator of the activin/TGF-β signaling pathway is selected from the group consisting of TGFβ1, TGFβ2, TGFβ3, activin A, activin B, activin AB or nodal, preferably the activator of activin/TGF-β signaling pathway is TGFβ3.

22. Method of any one of the preceding items, wherein the differentiation medium further comprises a cAMP analogue.

23. Method of any one of the preceding items, wherein the cAMP analogue is selected from the group consisting of forskolin, 8-(4-chloro-phenylthio)-2′-O-methyladenosine-3′,5′-cyclic monophosphate (8CPT-2Me-cAMP), 8-Chloro-cAMP (8-Cl-cAMP), Bucladesine, Rp-adenosine .3., 5.,-cyclic monophosphorothioate sodium salt (Rp-cAMPS), Sp-8-hydroxyadenosine .3., 5.,-cyclic monophosphorothioate sodium salt (Sp-80H-cAMPS) and Rp8-hydroxyadenosine .3., 5.,-cyclic monophosphorothioate sodium salt (Rp-80H-cAMPS) or dbcAMP, preferably the cAMP analogue is dbcAMP.

24. Method of any one of the preceding items, wherein the differentiation medium is a N2B27 medium.

25. Method of any one of the preceding items, wherein the N2B27 medium comprises equal amounts of Neurobasal medium and DMEM/F12 medium.

26. Method of any one of the preceding items, wherein the differentiation medium further comprises penicillin and streptomycin, preferably at a concentration of 1%.

27. Method of any one of the preceding items, wherein the differentiation medium further comprises glutamine, preferably L-glutamine, more preferably L-glutamine at a concentration of 2mM.

28. Method of any one of the preceding items, wherein the differentiation medium further comprises B27 supplement without vitamin A, preferably at a concentration of 1:100 (supplement:medium).

29. Method of any one of the preceding items, wherein the differentiation medium further comprises N2 supplement, preferably at a concentration of 1:200 (supplement:medium).

30. Method of any one of the preceding items, wherein the differentiation medium does not comprise FGF8.

31. Method of any one of the preceding items, wherein the cells are kept for 1, 2, 3, 4, 5, 6, 7, 8, 9, 10, 11, 12 days, preferably for 6 days, in the differentiation medium of step a).

32. Method of any one of the preceding items, wherein the cells are kept for 42 days in the differentiation medium of step b).

33. Method of any one of the preceding items, wherein the differentiation medium is changed every 2 days or 4 days.

34. Method of any one of the preceding items, wherein NESC cells are maintained in a maintenance medium before differentiation.

35. Method of any one of the preceding items, wherein the maintenance medium comprises
(i) a SHH-pathway activator;
(ii) canonical WNT-signaling activator; and
(iii) an antioxidant.

36. Method of any one of the preceding items, wherein the maintenance medium comprises N2B27 medium.

37. Method of any one of the preceding items, wherein the canonical WNT-signaling activator is selected from the group consisting of Norrin, R-spondin 2 or WNT protein.

38. Method of any one of the preceding items, wherein canonical WNT-signaling activator blocks Axin or APC e.g. via siRNA.

39. Method of any one of the preceding items, wherein canonical WNT-signaling activator is a GSK-3 inhibitor.

40. Method of any one of the preceding items, wherein the GSK-3 inhibitor is selected from the group consisting of CHIR 99021, SB-216763, 6-bromoindirubin-3′-oxime, Tideglusib, GSK-3 inhibitor 1, AZD1080, TDZD-8, TWS119, CHIR-99021 HCl, CHIR-98014, SB 415286, LY2090314, AR-A014418 or IM-12.

41. Method of any one of the preceding items, wherein the GSK-3 inhibitor is CHIR 99021.

42. Method of any one of the preceding items, wherein the maintenance medium is used to prepare NESCs at a density of 16000 cells per 96-well plate well.

43. Method of any one of the preceding items, wherein the NESCs are contacted with a dilution ratio of 1:15 of Matrigel and maintenance medium.

44. Method of any one of the preceding items, wherein the cells are kept for at least 6, 12, 18, 24, 48 hours, preferably 24 hours in the maintenance medium.

45. Method of any one of the preceding items, wherein cells are kept for 6 days in the differentiation medium as defined in a) of item 1.

46. Method of any one of the preceding items, wherein cells are kept for 21 days in the differentiation medium as defined in b) of item 1.

47. Method of any one of the preceding items, wherein molecule of interest is a siRNA, miRNA, binding molecule, small molecule or compound.

48. Method for producing dopaminergic neurons in a three-dimensional cell culture, the method comprising
a) contacting neuroepithelial stem cells (NESCs) with a matrix or a scaffold and optionally further a maintenance medium;
b) plating the NESCs in a container, wherein the container does not comprise a mean for generating fluid flow, thereby forming a three dimensional gel comprising NESCs;
c) differentiating NESCs obtained in b) in a differentiation medium, wherein the differentiation medium comprises
(i) a SHH-pathway activator;
(ii) at least two different neurotrophins; and
(iii) an antioxidant; and
d) further differentiating the cells obtained in c) in a differentiation medium, wherein the differentiation medium comprises
(i) at least two different neurotrophins; and
(ii) an antioxidant;
thereby differentiating said NESCs into dopaminergic neurons.

49. The method of item 48, wherein the container does not comprise an electronic device.

50. The method of item 49, wherein the electronic device is a pump.

51. The method of item 48-50, wherein the container does not comprise a mechanic element.

52. The method of item 51, wherein the mechanic element is an enclined plane.

53. The method of item 48-52, wherein the differentiation is not performed in a microfluidic cell culture.

54. The method of any of the preceding items, wherein the three-dimensional cell culture is not an organoid (cell) culture or not a midbrain organoid culture.

55. The method of any of the preceding items, wherein in the three-dimensional cell culture at least about 25%, preferably at least about 80% of all cells are neurons.

56. The method of any of the preceding items, wherein in the number of neurons is determined by a ratio of DAPI positive cells (for all cells present in the cell culture) and Tuj1-expressing cells (which are mature neurons).

57. The method of any of the preceding items, wherein in the three-dimensional cell culture less than about 50% of all neurons are dopaminergic neurons.

58. The method of any of the preceding items, wherein in the number of dopaminergic neurons within all neurons is determined by a ratio of Tuj1-expressing cells (all neurons) and Tuj1 and tyrosine hydroxylase (TH) expressing cells (dopaminergic neurons).

59. The method of any of the preceding items, wherein the expression of Tuj1 and/or TH is determined by immunohistochemistry.

60. Method for segmenting an image of a cell culture, wherein the cells within the cell culture have been dyed with at least a first dye (e.g. TH) selectively staining a specific neuronal subtype, optionally dopaminergic neural cells, a second dye (Hoechst) selectively staining nuclei of cells and a third dye (e.g. Tuj1) selectively staining two or more neuronal subtypes, optionally all neuronal cells, wherein the fluorescence of each dye is spectrally separated from the fluorescence of the other two dyes, the method comprising:
identifying a first group of pixels (THMask) in the image of the cell culture by low-pass filtering (size=10, sigma=1; THLP) the spectral content of the image corresponding to the first dye and by and discarding pixels with values lying below a first threshold (threshold=100);
identifying a second group of pixels (NucleiMask) in the image of the cell culture by applying a first low-pass filter (size=10, sigma=2; foreground in NucleiDoG) to the spectral content of the image corresponding to the second dye, subtracting therefrom low-pass filtered spectral content (background in NucleiDoG) of the image corresponding to the second dye with a second low-pass filter (size=60, signal=20), wherein the size and standard deviation of the first filter are smaller than the size and standard deviation of the first filter, and discarding pixels with values lying below a second threshold (threshold=10, NucleiDoGmask); identifying a first subgroup of pixels (Tuj1LocalMask) in the image of the cell culture by applying a third low-pass filter (size=10, sigma=3, foreground in Tuj1DoG) to the spectral content of the image corresponding to the third dye, subtracting therefrom low-pass filtered spectral content (background in Tuj1DoG) of the image corresponding to the third dye with a fourth low-pass filter (size=20, sigma=6), wherein the size and standard deviation of the third filter are smaller than the size and standard deviation of the fourth filter, and discarding pixels with values lying below a third threshold (threshold=3, Tuj1LocalMask), identifying a second subgroup of pixels (Tuj1GlobalMask) in the image of the cell culture by low-pass filtering (size=10, sigma=3; Tuj1LP) the spectral content of the image corresponding to the third dye and discarding pixels with values lying below a fourth threshold (threshold=150, Tuj1GlobalMask);
identifying a third group of pixels (NeuroMask) by joining the first subgroup of pixels and the second subgroup of pixels and discarding therefrom pixels which are part of the second group of pixels.

61. Method of item 54, wherein identifying the second group of pixels further comprises discarding pixels forming connected structures which comprise less than a first number of pixels (removal of connected components with <200 px in Nuclei Mask).

62. Method of item 60 or 61, wherein identifying the third group of pixels further comprises discarding pixels forming connected structures which comprise less than the first number of pixels (removal of connected components with <200 px in NeuroMask).

63. Dopaminergic neuron obtainable by a method of any one of items 48-59.

64. Dopaminergic neuron of item 63 for use in the treatment of a subject, preferably a subject suffering from a neurodegenerative disease.

The present invention also relates to dopaminergic neurons obtainable or obtained by the methods of the present invention. Such a dopaminergic neuron can express tyrosine hydroxylase (TH). Tyrosine hydroxylase is the enzyme responsible for catalyzing the conversion of the amino acid L-tyrosine to L-3,4-dihydroxyphenylalanine (L-DOPA). L-DOPA is a precursor for dopamine. This neurotransmitter is present in dopaminergic neurons. Additionally the dopaminergic neuron can express TUBplll. TUβIII can also be referred to as Tuj1 and is expressed specifically by neurons.

The present invention also relates to the dopaminergic neuron as described herein for use in treatment of a disease. Treatable diseases include neurodegenerative diseases. Exemplary neurodegenerative disease to be treated are Alzheimer's disease or Parkinson's disease. Thus, the dopaminergic neuron as described herein can be used in treatment of Parkinson's disease.

TABLE 1
Sequences referred to herein

| | | |
|---|---|---|
| 1 | P07101 | MPTPDATTPQAKGFRRAVSELDAKQAEAIMVRGQGAPGPSLTGSPWPGTAAPAA |
| | HUMAN | SYTPTPRSPRFIGRRQSLIEDARKEREAAVAAAAAAVPSEPGDPLEAVAFEEKEGKA |
| | Tyrosine | VLNLLFSPRATKPSALSRAVKVFETFEAKIHHLETRPAQRPRAGGPHLEYFVRLEVRR |
| | 3-mono- | GDLAALLSGVRQVSEDVRSPAGPKVPWFPRKVSELDKCHHLVTKFDPDLDLDHPG |
| | oxygenase | FSDQVYRQRRKLIAEIAFQYRHGDPIPRVEYTAEEIATWKEVYTTLKGLYATHACGE |
| | | HLEAFALLERFSGYREDNIPQLEDVSRFLKERTGFQLRPVAGLLSARDFLASLAFRVF |
| | | QCTQYIRHASSPMHSPEPDCCHELLGHVPMLADRTFAQFSQDIGLASLGASDEEIE |
| | | KLSTLYWFTVEFGLCKQNGEVKAYGAGLLSSYGELLHCLSEEPEIRAFDPEAAAVQP |
| | | YQDQTYQSVYFVSESFSDAKDKLRSYASRIQRPFSVKFDPYTLAIDVLDSPQAVRRSL |
| | | EGVQDELDTLAHALSAIG |
| 2 | NM_199293.2 | CGGACCTCCACACTGAGCCATGCCCACCCCCGACGCCACCACGCCACAGGCCAA |
| | Homo sapiens | GGGCTTCCGCAGGGCCGTGTCTGAGCTGGACGCCAAGCAGGCAGAGGCCATC |
| | tyrosine | ATGGGCGCCCCGGGGCCCAGCCTCACAGGCTCTCCGTGGCCTGGAACTGCAGC |
| | hydroxylase | CCCAGCTGCATCCTACACCCCCACCCCAAGGTCCCCGCGGTTCATTGGGCGCAG |
| | (TH), | GCAGAGCCTCATCGAGGACGCCCGCAAGGAGCGGGAGGCGGCGGTGGCAGC |
| | transcript | AGCGGCCGCTGCAGTCCCCTCGGAGCCCGGGGACCCCCTGGAGGCTGTGGCCT |
| | variant | TTGAGGAGAAGGAGGGGAAGGCCGTGCTAAACCTGCTCTTCTCCCCGAGGGCC |
| | 3, mRNA | ACCAAGCCCTCGGCGCTGTCCCGAGCTGTGAAGGTGTTTGAGACGTTTGAAGC |
| | | CAAAATCCACCATCTAGAGACCCGGCCCGCCCAGAGGCCGCGAGCTGGGGGCC |
| | | CCCACCTGGAGTACTTCGTGCGCCTCGAGGTGCGCCGAGGGGACCTGGCCGCC |
| | | CTGCTCAGTGGTGTGCGCCAGGTGTCAGAGGACGTGCGCAGCCCCGCGGGGC |
| | | CCAAGGTCCCCTGGTTCCCAAGAAAAGTGTCAGAGCTGGACAAGTGTCATCAC |
| | | CTGGTCACCAAGTTCGACCCTGACCTGGACTTGGACCACCCGGGCTTCTCGGAC |
| | | CAGGTGTACCGCCAGCGCAGGAAGCTGATTGCTGAGATCGCCTTCCAGTACAG |
| | | GCACGGCGACCCGATTCCCCGTGTGGAGTACACCGCCGAGGAGATTGCCACCT |
| | | GGAAGGAGGTCTACACCACGCTGAAGGGCCTCTACGCCACGCACGCCTGCGGG |
| | | GAGCACCTGGAGGCCTTTGCTTTGCTGGAGCGCTTCAGCGGCTACCGGGAAGA |
| | | CAATATCCCCCAGCTGGAGGACGTCTCCCGCTTCCTGAAGGAGCGCACGGGCTT |
| | | CCAGCTGCGGCCTGTGGCCGGCCTGCTGTCCGCCCGGGACTTCCTGGCCAGCCT |
| | | GGCCTTCCGCGTGTTCCAGTGCACCCAGTATATCCGCCACGCGTCCTCGCCCAT |
| | | GCACTCCCCTGAGCCGGACTGCTGCCACGAGCTGCTGGGGCACGTGCCCATGC |
| | | TGGCCGACCGCACCTTCGCGCAGTTCTCGCAGGACATTGGCCTGGCGTCCCTGG |
| | | GGGCCTCGGATGAGGAAATTGAGAAGCTGTCCACGCTGTACTGGTTCACGGTG |
| | | GAGTTCGGGCTGTGTAAGCAGAACGGGGAGGTGAAGGCCTATGGTGCCGGGC |
| | | TGCTGTCCTCCTACGGGGAGCTCCTGCACTGCCTGTCTGAGGAGCCTGAGATTC |
| | | GGGCCTTCGACCCTGAGGCTGCGGCCGTGCAGCCCTACCAAGACCAGACGTAC |
| | | CAGTCAGTCTACTTCGTGTCTGAGAGCTTCAGTGACGCCAAGGACAAGCTCAG |
| | | GAGCTATGCCTCACGCATCCAGCGCCCCTTCTCCGTGAAGTTCGACCCGTACAC |
| | | GCTGGCCATCGACGTGCTGGACAGCCCCCAGGCCGTGCGGCGCTCCCTGGAGG |
| | | GTGTCCAGGATGAGCTGGACACCCTTGCCCATGCGCTGAGTGCCATTGGCTAG |
| | | GTGCACGGCGTCCCTGAGGGCCCTTCCCAACCTCCCCTGGTCCTGCACTGTCCC |
| | | GGAGCTCAGGCCCTGGTGAGGGGCTGGGTCCCGGGTGCCCCCCATGCCCTCCC |
| | | TGCTGCCAGGCTCCCACTGCCCCTGCACCTGCTTCTCAGCGCAACAGCTGTGTG |
| | | TGCCCGTGGTGAGGTTGTGCTGCCTGTGGTGAGGTCCTGTCCTGGCTCCCAGG |
| | | GTCCTGGGGGCTGCTGCACTGCCCTCCGCCCTTCCCTGACACTGTCTGCTGCCCC |
| | | AATCACCGTCACAATAAAAGAAACTGTGGTCTCTA |
| 3 | Q13509 | MREIVHIQAGQCGNQIGAKFWEVISDEHGIDPSGNYVGDSDLQLERISVYYNEASS |
| | TBB3_HUMAN | HKYVPRAILVDLEPGTMDSVRSGAFGHLFRPDNFIFGQSGAGNNWAKGHYTEGA |
| | Tubulin | ELVDSVLDVVRKECENCDCLQGFQLTHSLGGGTGSGMGTLLISKVREEYPDRIMNT |
| | beta-3 chain | FSVVPSPKVSDTVVEPYNATLSIHQLVENTDETYCIDNEALYDICFRTLKLATPTYGDL |
| | | NHLVSATMSGVTTSLRFPGQLNADLRKLAVNMVPFPRLHFFMPGFAPLTARGSQ |
| | | QYRALTVPELTQQMFDAKNMMAACDPRHGRYLTVATVFRGRMSMKEVDEQML |
| | | AIQSKNSSYFVEWIPNNVKVAVCDIPPRGLKMSSTFIGNSTAIQELFKRISEQFTAMF |
| | | RRKAFLHWYTGEGMDEMEFTEAESNMNDLVSEYQQYQDATAEEEGEMYEDDEE |
| | | ESEAQGPK |

### EXAMPLES

### Example 1

### Methods

Outline of the Study

Human neural epithelial cells (hNESC), directly derived from iPSCs, were generated as previously described (Reinhardt et al., Derivation and expansion using only small molecules of human neural progenitors for neurodegenerative disease modelling. PLoS One 2013, 8(11)).

One line derived from an healthy individual and one line derived from a PD patient were used. Each line had an isogenic control where the mutation was either inserted or corrected. After 48-72h from the seeding, hNESC were differentiated into neurons for 6 weeks. An overview of the study outline is given in FIG. 1.

Cell Culture

hNESC were cultured in N2B27 medium (DMEM-F12 (Gibco)/Neurobasal (Gibco) 50:50 supplemented with 1:200 N2 supplement (Invitrogen), 1:100 B27 supplement lacking vitamin A (Invitrogen), penicillin/streptomycin and glutamine (Invitrogen) supplemented with 3 μM CHIR 99021 and 0.5 μM PMA and 150 ascorbic acid. Cells were maintained in Matrigel coated plates. At a confluence of 70-80% cells were detached using Accutase for 3′, collected by centrifugation and the appropriate amount of cells were seeded in the different conditions (see below). Neuronal differentiation was achieved by a media containing N2B27 Medium supplemented with 10 ng/mL BDNF (Peprotech), 10 ng/mL GDNF (Peprotech), 1 ng/mL TGF-β3 (Peprotech), 200 μM ascorbic acid and 500 μM dbcAMP (Sigma Aldrich). For the first 6 days, 1 μM PMA was also be added. Neurons were kept in culture for 6 weeks. Differentiation media with PMA was changed every two days for the first 6 days. From day 7 onwards, the media without PMA was changed every 4th day. For the phenotype rescue experiments, the LRRK2 kinase inhibitor Inh2 (CZC-25146, Millipore) at 0.5 μM concentration or the vehicle (DMSO) were added freshly to the media every two days for the first 6 days and every 4 days afterwards. Notably, in the three-dimensional cell cultures at least 80% of all cells are neurons.

2D Cultures

hNESC were seeded on Matrigel-coated optic clear 96 well plates (Perkin Elmer) at a density of 10,000 cells per well. After 24h, neuronal differentiation was induced.

3D Cultures in Thin Layer of Matrigel

hNESC were suspended in Matrigel and media (1:15 dilution ratio) and vortexed for 20 s. 16,000 cells per well were transferred into Optilux Black/Clear bottom 96-well plates (100 μl in each well, BD Biosciences) using pre-chilled pipettes. The plates were incubated for 1 h at 37° C. to form thin-layer (100-300 μm) 3D gels at the bottom of the plates. After 24 h, neuronal differentiation was induced.

Immunofluorescence Staining

2D and 3D cultures were fixed with 4% PFA 40 minutes and overnight at 4° C. respectively. After 3 washes in PBS cells were permeabilized 15 min in 0.3% Triton in PBS. For the 3D cultures, the permeabilization and blocking were performed for 6 h at room temperature: BSA 2%, NGS 2%, Glycine 0.3 M, and Triton 0.3% in PBS. After blocking for 1 h, the first antibodies were incubated for 24 h at room temperature. The combination of secondary antibodies was then added for additional 2 h. Cells were then analyzed by the neuronal marker Tuj1 (Millipore, AB9354), the dopaminergic marker TH (Santa Cruz, sc-14007), pS129αsynuclein (Abcam, ab184674), and the dye Hoechst.
The main read-outs of the immunofluorescence analysis are the following:
1) percentage of TH+ neurons over total number of neurons (Tuj1);
2) fragmentation of TH+ neurons as a sign of neurodegeneration;
3) semi-quantitative analysis of the levels of pS129αsynuclein (pS129SNCA) over total number of neurons (Tuj1).

Image Acquisition

The image acquisition for all the conditions was performed using the same parameters with the high content screening microscope Opera (PerkinElmer) at 20x lens magnification. Skew analysis was performed before every plate acquisition.

Data Analysis

The acquired images were analyzed with a battery of scripts developed in-house with Matlab Software. How this software operates is further described in Example 3. All the features for each treatment were normalized to the line with the WT-LRRK2 within each pair. The lines with the WT-LRRK2 represent the 100% for the respective pairs. Data are represented by bar plots generated in GraphPad. The statistical analysis was performed with one-sample t test to compare LRRK2G2019S vs WT-LRRK2.

### Example 2

### Results

Our data shows that 3D cultivation systems are providing brain tissue-like environment which allows to measure neuronal phenotype which can be missed in classical 2D cultures. We found that our Matrigel-based 3D culture condition strongly exacerbates G2019S-LRRK2 related phenotype as shown by the decreased expression of TH in the PD patient compared to the isogenic pair where the mutation was corrected (PDGC) FIG. 2A. We also observed signs of dopaminergic degeneration (B) represented by TH fragmentation along swollen dendrites and/or axon in PD derived cells compared to PDGC. Also, the levels of pS129synuclein significantly decreased in cells derived from healthy individual where G2019S-LRRK2 mutation was inserted (HMut) compared to the isogenic pair H with WT-LRRK2.

These phenotypes were completely missed in 2D conditions. Since we used the same Matrigel for 2D and 3D differentiation protocols, these differences are not to be related to the matrix composition but only to the 3D system. 3D models can be easily adapted for high content high throughput image analysis for drug screening as robust phenotypes, recapitulating key features of the human disease, can be identify.

We then tested the effect of the LRRK2 kinase Inh2 to rescue the described phenotype. FIG. 3 shows a heat map of a cell line pair, derived from an healthy individual, which allows to rapidly visualize the alteration of features from the WT to the G2019S mutant line and evaluate the rescue of the drug.

The annotated clustering shows the similarities between the conditions based on the assessment of all the features analyzed, indicated on the right side of the heat map. The appearance of the phenotype in the mutant line is indicated by the different color code compared to the WT. The rescue of the phenotype in shown by the co-clustering of WT and mutant lines upon Inh2 treatment. Interestingly, the PD patient derived line denominated IM5 (FIG. 4) also showed a phenotype compared to the same cell line where the mutation was inserted but there was no rescue of the phenotype. In fact, the mutant line is clustering together independently of the treatment (Inh2 or DMSO as vehicle) and the same applies to the WT line. This different behavior nicely highlights the contribution of the genetic background of the patient from which the cells are originated to the outcome of a certain pharmacological intervention. It captures the goal of developing a personalized screening platform where drugs can be pre-tested before reaching the patient.

FIG. 5 shows the reduction in the levels of pS129-α-SNCA in HMut compare to H and the rescue after Inh2 administration in 3D conditions (A). The same lines failed to show analogous behavior in 2D conditions (B).

### Example 3

### Image Analysis: Immunofluorescence

The image acquisition of human neural epithelial stem cell (hNESCs) derived neuronal cultures was performed with the high content screening microscope Opera (PerkinElmer) at 20x lens magnification. Skew analysis was performed before every plate acquisition.

Immunofluorescence 4 channel 3D images of neuronal cultures were analysed in Matlab (Version 2016a, Mathworks). The developed custom image analysis algorythm automates 3 major steps, namely: segmentation of nuclei and neurons, and feature extraction (FIG. 6).

Image preprocessing for the segmentation of nuclei was done via a difference of gaussians (NucleiDoG). Briefly, a foreground image was computed by convolving the raw Hoechst channel with a gaussian filter of size 10 and standard deviation 2. Similarly, for the background image, a gaussian filter of size 60 and standard deviation 20 was used. The difference was computed by substracting the background from the foreground. The first rough nuclei mask was defined by those pixels with graytone values larger than 10 (NucleiDoGmask). Only connected components with at least 200 pixels were retained (NucleiMask).

For the segmentation of neurons a strategy combining global and local thresholding was implemented. For global thresholding, image preprocessing was done via low pass filtering (Tuj1 LP). For this purpose, the raw Tuj1 channel was convolved with a gaussian filter of size 10 and standard deviation 3. The global neuronal mask is defined by threshold 150 (Tuj1GlobalMask). For local thresholding, a difference of gaussians was applied in the preprocessing step (Tuj1DoG). Precisely, the background defined via convolution with a gaussian filter of size 20 and standard deviation 6 was substracted from the foreground defined via convolution with a gaussian filter of size 10 and standard deviation 3. The local neuronal mask is defined by those pixels with values larger than 3 (Tuj1LocalMask). The concepts of global and local thresholding were combined by retaining those pixels in the neuronal mask which were detected by at least one of these methods. For refining the neuronal mask, pixels overlapping with the NucleiMask and connected components with less than 200 pixels were removed (NeuroMask).

For the analysis of the TH channel, an additional mask was defined by preprocessing the raw TH channel via convolution with a gaussian filter of size 10 and standard deviation 1 (THLP), and thresholding by pixel value 100 (THMask).

In a similar manner also the further data were analyzed.

TABLE 1
Features from immunofluorescence

| Feature | Description |
|---|---|
| sumNucMask | Count of nuclei mask pixels |
| sumNeuroMask | Count of neuronal mask pixels |
| TH_ProportionInTuj1 | Sum of neuronal TH pixel |
| | intensities/sumNeuroMask |
| Sum_Hoechst | Sum of raw Hoechst pixel values within |
| | the nucleus mask |
| Sum_TH | Sum of raw TH pixel values within the |
| | neuronal mask |
| Sum_Tuj1 | Sum of raw Tuj1 pixel values within the |
| | neuronal mask |

The work leading to this invention was funded by the Luxembourg Fonds National de Recherche (FNR) under grant No. FNR/PoC/11180855.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

All publications and patents cited in this disclosure are incorporated by reference in their entirety. To the extent the material incorporated by reference contradicts or is inconsistent with this specification, the specification will supersede any such material.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

When used herein, the term “about” is understood to mean that there can be variation in the respective value or range (such as pH, concentration, percentage, molarity, number of amino acids, time etc.) that can be up to 5%, up to 10%, up to 15% or up to and including 20% of the given value. For example, if a formulation comprises about 5 mg/ml of a compound, this is understood to mean that a formulation can have between 4 and 6 mg/ml, preferably between 4.25 and 5.75 mg/ml, more preferably between 4.5 and 5.5 mg/ml and even more preferably between 4.75 and 5.25 mg/ml, with the most preferred being 5 mg/ml. As used herein, an interval which is defined as “(from) X to Y” equates with an interval which is defined as “between X and Y”. Both intervals specifically include the upper limit and also the lower limit. This means that for example an interval of “5 mg/ml to 10 mg/ml” or “between 5 mg/mI and 10 mg/ml” includes a concentration of 5, 6, 7, 8, 9, and 10 mg/ml as well as any given intermediate value.

### REFERENCES

Antoni D, Burckel H, Josset E, Noel G. Three-Dimensional Cell Culture: A Breakthrough in Vivo. Int J Mol Sci. 2015 Mar. 11; 16(3):5517-5527.
Bertram and Tanzi (2005) “The genetic epidemiology of neurodegenerative disease” J Clin Invest. 115(6): 1449-1457.
Bouhouche (2017) “LRRK2 G2019S Mutation: Prevalence and Clinical Features in Moroccans with Parkinson's Disease” Parkinson's Disease Volume 2017, p.1-7.
Carl-Henrik Heldin, Kohei Miyazono & Peter ten Dijke TGF-bold beta signalling from cell membrane to nucleus through SMAD proteins. Nature 390, 465-471 (4 Dec. 1997).
Choudhry Z, Rikani A A, Choudhry A M, Tariq S, Zakaria F, Asghar M W, Sarfraz M K, Haider K, Shafiq A A, Mobassarah N J. Sonic hedgehog signalling pathway: a complex network. Ann Neurosci. 2014 Jan.; 21 (1):28-31.
Fimia G M, Sassone-Corsi P. Cyclic A M P signalling. J Cell Sci. 2001 Jun.; 114(Pt 11):1971-2.
Gilks et al. (2005), The Lancet 365 (9457): 415-416.
Goldwurm et al. (2005) “The G6055A (G2019S) mutation in LRRK2 is frequent in both early and late onset Parkinson's disease and originates from a common ancestor” Med Genet; 42; doi: 10.1136; jmg.2005.035568, pages 1-8.
Jo et al. (2016) “Midbrain-like organoids from human pluripotent stem cells contain functional dopaminergic and neuromelanin-producing neurons” Cell Stem Cell 19, 248-257.
Klimanskaya I. Embryonic stem cells from blastomeres maintaining embryo viability. Semin Reprod Med. 2013 Jan.; 31 (1):49-55.
Lancaster et al. (2013) “Cerebral organoids model human brain development and microcephaly” Nature 501, 373-379 (cerebral organoid).
Lee and Chung (2016) “Integrating Gene Correction in the Reprogramming and Transdifferentiation Processes: A One-Step Strategy to Overcome Stem Cell-Based Gene Therapy Limitations” Stem Cells Int.: 2725670.
Logan and Nusse Annu. Rev. Cell Dev. Biol. (2004) 20:781-810.
Monzel et al. (2017) “Derivation of human midbrain-specific organoids from neuroepithelial stem cells” Stem Cell Reports vol. 8, 1144-1154
Nordberg J, Amér ES. Reactive oxygen species, antioxidants, and the mammalian thioredoxin system. Free Radic Biol Med. 2001 Dec. 1; 31 (11):1287-312.
Quian et al. (2016) “Brain-region-specific organoids using mini-bioreactors for modeling ZIKV exposure” Cell 165, 771-785.
Ravi M, Paramesh V, Kaviya S R, Anuradha E, Solomon F D. 3D cell culture systems: advantages and applications. J Cell Physiol. 2015 January; 230(1):16-26.
Reinhardt P, Glatza M, Hemmer K, Tsytsyura Y, Thiel C S, Höing S, Moritz S, Parga J A, Wagner L, Bruder J M, Wu G, Schmid B, Röpke A, Klingauf J, Schwamborn J C, Gasser T, Schöler HR, Sterneckert J. Derivation and expansion using only small molecules of human neural progenitors for neurodegenerative disease modeling. PLoS One. 2013; 8(3):e59252.
Reinhardt et al. (2013) “Genetic correction of a LRRK2 mutation in human iPSCs links parkinsonian neurodegeneration to ERK-dependent changes in gene expression.” Cell Stem Cell. 12(3):354-67.

Takahashi K, Tanabe K, Ohnuki M, Narita M, Ichisaka T, Tomoda K, Yamanaka S. Induction of pluripotent stem cells from adult human fibroblasts by defined factors.
Cell. 2007 Nov 30.; 131(5):861-72.
Takahashi, K; Yamanaka, S “Induction of pluripotent stem cells from mouse embryonic and adult fibroblast cultures by defined factors”. Cell 2006, 126 (4): 663-76).
Tanabe K. Takahashi K. Yamanaka S. Induction of pluripotency by defined factors. Proc. Jpn. Acad., 2014, Ser. B 90.

1. A method for identifying molecules promoting or inhibiting dopaminergic neuronal differentiation and/or death of dopaminergic neurons in a three-dimensional cell culture, the method comprising
a) differentiating neuroepithelial stem cells (NESCs) in a differentiation medium, wherein the differentiation medium comprises
(i) a sonic hedgehog (SHH)-pathway activator;
(ii) at least two different neurotrophins, and
(iii) an antioxidant;
b) further differentiating the cells obtained in a) in a differentiation medium, wherein the differentiation medium comprises
(i) at least two different neurotrophins, and
(ii) an antioxidant; and
c) adding a molecule of interest to the differentiation medium in a) and/or b),
wherein an increase of the differentiation into dopaminergic neurons compared to a control indicates that the molecule of interest promotes dopaminergic neuronal differentiation and/or inhibits death of dopaminergic neurons and wherein a decrease of the differentiation into dopaminergic neurons compared to a control indicates that the molecule of interest inhibits dopaminergic neuronal differentiation and/or induces death or neurodegeneration of dopaminergic neurons.

2. The method of claim 1, wherein the differentiation into dopaminergic neurons is measured by measuring the expression of tyrosine hydroxylase (TH) or by measuring the expression of TH among class III β-tubulin (Tujl-)positive neurons.

3. The method of claim 1, wherein the death of dopaminergic neurons is measured by measuring fragmentation of TH-positive neurons.

4. The method of claim 1, wherein the NESC is obtained from an induced pluripotent stem cell (iPSC).

5. The method of claim 4, wherein the iPSCs have been obtained from a peripheral blood Mononuclear Cells (PBMCs) from blood, keratinocyte, T-cell, CD34+ cell, myeloid cell, or a renal epithelial cell or fibroblasts.

6. The method of claim 4, wherein the iPSCs have been obtained from a subject suffering from a neurodegenerative disease.

7. The method of claim 6, wherein the neurodegenerative disease is selected from the group consisting of Alzheimer's disease, Parkinson's disease, amyotrophic lateral sclerosis, Huntington's disease and frontotemporal dementia.

8. The method of claim 1, wherein the differentiation medium in b) does not comprise a SHH-pathway activator.

9. The method of claim 1, wherein the differentiation medium does not comprise fibroblast growth factor 8 (FGF8).

10. The method of claim 1, wherein molecule of interest is a siRNA, miRNA, binding molecule, small molecule or compound.

11. A method for producing dopaminergic neurons in a three-dimensional cell culture, the method comprising
a) contacting neuroepithelial stem cells (NESCs) with a matrix and/or a scaffold and optionally further a maintenance medium;
b) plating the NESCs in a container, wherein the container does not comprise a mean for generating fluid flow, thereby forming a three dimensional gel comprising NESCs;
c) differentiating NESCs obtained in b) in a differentiation medium, wherein the differentiation medium comprises
(i) a SHH-pathway activator;
(ii) at least two different neurotrophins; and
(iii) an antioxidant;
d) further differentiating the cells obtained in c) in a differentiation medium, wherein the differentiation medium comprises
(i) at least two different neurotrophins; and
(ii) an antioxidant;
thereby differentiating said NESCs into dopaminergic neurons.

12. The method of claim 11, wherein the container does not comprise an electronic device and/or mechanic element.

13. The method of claim 12, wherein the electronic device is a pump.

14. A dopaminergic neuron obtainable by a method of claim 11.

15. The dopaminergic neuron of claim 14 for use in the treatment of a disease.

16. The method of claim 11, wherein the three-dimensional cell culture is not an organoid culture.

17. The method of claim 11, wherein the three-dimensional cell culture is not a midbrain organoid culture.

18. The method of claim 11, wherein in the three-dimensional cell culture at least about 25%, of all cells are neurons.

19. The method of claim 11, wherein in the three-dimensional cell culture less than about 50% of all neurons are dopaminergic neurons.
